(19) United States
(12) Patent Application Publication
(10) Pub. No.: US 2022/0053834 A1
(43) Pub. Date: Feb. 24, 2022

(54) VAPORIZATION DEVICE WITH RESIDUE PREVENTION OR REDUCTION

(71) Applicant: HEXO OPERATIONS INC., Ottawa (CA)

(72) Inventors: Patrick WOODS, Ottawa (CA); Max ALSAYAR, Ottawa (CA); Guy DEGRACE, Ottawa (CA)

(21) Appl. No.: 17/416,706

(22) PCT Filed: Dec. 20, 2019
(86) PCT No.: PCT/CA2019/051881

§ 371 Date: Jun. 21, 2021

Related U.S. Application Data
(60) Provisional application No. 62/783,369, filed on Dec. 21, 2018.
(60) Provisional application No. 62/792,599, filed on Jan. 15, 2019.
(60) Provisional application No. 62/938,996, filed on Nov. 22, 2019.

(51) Int. Cl. A24F 40/485; A24F 40/46

(52) U.S. Cl. CPC A24F 40/485; A24F 40/46

(57) ABSTRACT

An apparatus includes a chamber to store a first vaporization substance, and an atomizer, in fluid communication with the chamber, to generate vapor from the vaporization substance by heating the vaporization substance. Some embodiments include an insulated channel, in fluid communication with the atomizer, to carry the vapor away from the atomizer. An auxiliary heater, to heat the vapor and/or to heat one or more components of the apparatus, could also or instead be provided. A cooler to cool the vapor is provided in some embodiments.

### CROSS-REFERENCE TO RELATED APPLICATIONS

This application is related to, and claims priority to, U.S. Provisional Patent Application No. 62/783,369, entitled “APPARATUS AND METHODS FOR SERIAL CONFIGURATIONS OF MULTI-CHAMBER VAPORIZATION DEVICES”, and filed on Dec. 21, 2018; U.S. Provisional Patent Application No. 62/792,599, entitled “VAPORIZATION DEVICE WITH RESIDUE PREVENTION OR REDUCTION”, and filed on Jan. 15, 2019; and U.S. Provisional Patent Application No. 62/938,996, entitled “VAPORIZATION DEVICE WITH VAPOR COOLING”, and filed on Nov. 22, 2019, the entire contents of each of which are incorporated by reference herein.

### FIELD

This application relates generally to vaporization devices, and in particular to vaporization devices with features intended to prevent or reduce buildup of residue from vaporization substances.

### BACKGROUND

A vaporization device is used to vaporize substances for inhalation. These substances are referred to herein as vaporization substances, and could include, for example, cannabis products, tobacco products, herbs, and/or flavorants. In some cases, substances in cannabis, tobacco, or other plants or materials extracted to generate concentrates are used as vaporization substances. These substances could include cannabinoids from cannabis, and nicotine from tobacco. In other cases, synthetic substances are artificially manufactured. Terpenes are common flavorant vaporization substances, and could be generated from natural essential oils or artificially.

Vaporization substances could be in the form of loose leaf in the case of cannabis, tobacco, and herbs, for example, or in the form of concentrates or derivative products such as liquids, waxes, or gels, for example. Vaporization substances, whether intended for flavor or some other effect, could be mixed with other compounds such as propylene glycol, glycerin, medium chain triglyceride (MCT) oil and/or water to adjust the viscosity of a final vaporization substance.

In a vaporization device, the vaporization substance is heated to the vaporization temperature of one or more constituents of the vaporization substance. This produces a vapor, which may also be referred to as an aerosol. The vapor is then inhaled by a user through a channel that is provided in the vaporization device, and often through a hose or pipe that is part of or attached to the vaporization device.

### SUMMARY

According to an aspect of the present disclosure, an apparatus includes a chamber to store a vaporization substance; an atomizer, in fluid communication with the chamber, to generate vapor from the vaporization substance by heating the vaporization substance; and an insulated channel, in fluid communication with the atomizer, to carry the vapor away from the atomizer.

In some embodiments, the insulated channel includes a thermally insulative material.

The insulated channel could include a thermal insulator, located at an inside part and/or an outside part of the insulated channel. The thermal insulator could be removable. In an embodiment, the thermal insulator includes a coating on the insulated channel.

A cooler could be provided to cool the vapor. The cooler could be or include an active cooler such as a thermoelectric cooler, and/or a passive cooler such as a heat sink. The heat sink could be or include a removable heat sink element, which could be coupled to the apparatus by a releasable coupling or magnetically, for example.

The cooler could be or include a heat exchanger to transfer heat from the vapor to a heat sink. The heat sink could be or include a material inside the heat exchanger.

Examples of a heat sink include air and a liquid.

The cooler could be inside the insulated channel.

An apparatus could include a mouthpiece in fluid communication with the insulated channel. The mouthpiece is releasably coupled to the apparatus in some embodiments, by a threaded engagement or a friction fit engagement, for example.

The mouthpiece could be indirectly in fluid communication with the insulated channel, through a further channel.

Multiple mouthpieces could be in fluid communication with the insulated channel through respective further channels.

A heater, in fluid communication with the atomizer, could be provided to heat the vapor.

A heater could also or instead be provided to heat the insulated channel.

In some embodiments, a heater is provided to heat one or more components of the apparatus. The one or more components of the apparatus could include any one or more of: the insulated channel, and a regulator to control movement of the vaporization substance from the chamber to the atomizer.

According to another aspect of the present disclosure, an apparatus includes: a chamber to store a vaporization substance; an atomizer, in fluid communication with the chamber, to generate vapor from the vaporization substance by heating the vaporization substance; and an auxiliary heater.

The auxiliary heater could be or include a vapor heater to heat the vapor.

The auxiliary heater could also or instead include a component heater to heat one or more components of the apparatus. The one or more components of the apparatus could include any one or more of: a channel that is in fluid communication with the atomizer to carry the vapor away from the atomizer, and a regulator to control movement of the vaporization substance from the chamber to the atomizer.

An apparatus could include a cooler, in fluid communication with the atomizer, to cool the vapor. The cooler could be or include an active cooler such as a thermoelectric cooler, or a passive cooler such as a heat sink. The heat sink is a removable heat sink element in some embodiments, and could be coupled to the apparatus by a releasable coupling or magnetically, for example.

The cooler could be or include a heat exchanger to transfer heat from the vapor to a heat sink, which could include a material inside the heat exchanger.

A heat sink could be or include air or a liquid, for example.

In an embodiment, the cooler is inside a channel that is in fluid communication with the atomizer to carry the vapor away from the atomizer.

An apparatus could include a mouthpiece in fluid communication with the atomizer. The mouthpiece could be releasably coupled to the apparatus, by a threaded engagement or a friction fit engagement, for example.

An apparatus could include a channel in fluid communication with the atomizer, and the mouthpiece could be indirectly in fluid communication with the atomizer through the channel and a further channel.

In some embodiments, an apparatus includes multiple mouthpieces in fluid communication with the atomizer through respective channels.

A method according to another aspect of the present disclosure involves: providing a chamber to store a vaporization substance; providing an atomizer, in fluid communication with the chamber, to generate vapor from the vaporization substance by heating the vaporization substance; and providing an insulated channel in fluid communication with the atomizer, to carry the vapor away from the atomizer.

Providing an insulated channel could involve providing, as the insulated channel, a channel that includes a thermally insulative material.

Another option for providing an insulated channel involves providing a channel and providing a thermal insulator for the channel. Providing a thermal insulator for the channel could involve providing the thermal insulator at an inside part of the channel and/or at an outside part of the channel.

Providing a thermal insulator could also or instead involve providing, as the thermal insulator, a removable thermal insulator.

The thermal insulator could be provided as a coating on the channel.

A method could involve providing a cooler, in fluid communication with the insulated channel, to cool the vapor. Providing a cooler could involve providing an active cooler such as a thermoelectric cooler, and/or providing a passive cooler such as a heat sink.

Providing a heat sink could involve providing a removable heat sink element, such as a removable heat sink element that is attachable, by a releasable coupling or magnetically for example, to an apparatus that includes the chamber, the atomizer, and the insulated channel.

In an embodiment, providing a cooler involves providing a heat exchanger to transfer heat from the vapor to a heat sink. The heat sink could be or include a material inside the heat exchanger.

In embodiments that involve a heat sink, the heat sink could be or include air or a liquid, for example.

A cooler could be provided inside the insulated channel.

A method could involve providing a mouthpiece in fluid communication with the insulated channel. Providing a mouthpiece could involve providing a mouthpiece that is releasably attachable, by a threaded engagement or by a friction fit engagement for example, to an apparatus that includes the chamber, the atomizer, and the insulated channel.

A mouthpiece could be indirectly in fluid communication with the insulated channel through a further channel.

In some embodiments, a method involves providing multiple mouthpieces in fluid communication with the insulated channel through respective further channels.

A method could involve providing a heater, in fluid communication with the atomizer, to heat the vapor.

A method could involve providing a heater to heat the insulated channel.

In general, a method could involve providing a heater to heat one or more components of an apparatus that includes the chamber, the atomizer, and the insulated channel. The one or more components of the apparatus could include any one or more of: the insulated channel, and a regulator to control movement of the vaporization substance from the chamber to the atomizer.

A method according to another aspect of the present disclosure includes: providing a chamber to store a vaporization substance; providing an atomizer, in fluid communication with the chamber, to generate vapor from the vaporization substance by heating the vaporization substance; and providing an auxiliary heater.

Providing an auxiliary heater could involve providing a vapor heater to heat the vapor. Providing an auxiliary heater could also or instead involve providing a component heater to heat one or more components of an apparatus that includes the chamber, the atomizer, and the auxiliary heater. The one or more components of the apparatus could include any one or more of: a channel that is in fluid communication with the atomizer to carry the vapor away from the atomizer, and a regulator to control movement of the vaporization substance from the chamber to the atomizer.

A method could include providing a cooler, in fluid communication with the atomizer, to cool the vapor. Providing a cooler could involve providing an active cooler such as a thermoelectric cooler, and/or providing a passive cooler such as a heat sink.

Providing a heat sink could involve providing a removable heat sink element, which could involve providing a removable heat sink element that is attachable, by a releasable coupling or magnetically for example, to an apparatus that includes the chamber, the atomizer, and the auxiliary heater.

Providing a cooler could involve providing a heat exchanger to transfer heat from the vapor to a heat sink. The heat sink could be or include a material inside the heat exchanger.

In some embodiments, a heat sink could be or include air and/or a liquid.

Providing a cooler could involve providing the cooler inside a channel that is in fluid communication with the atomizer to carry the vapor away from the atomizer.

A method could involve providing a mouthpiece in fluid communication with the atomizer. Providing a mouthpiece could involve providing a mouthpiece that is releasably attachable, by a threaded engagement or by a friction fit engagement for example, to an apparatus that includes the chamber, the atomizer, and the auxiliary heater.

A method could involve providing a channel in fluid communication with the atomizer, in which case the mouthpiece could be indirectly in fluid communication with the atomizer through the channel and a further channel.

In an embodiment, a method includes providing multiple mouthpieces in fluid communication with the atomizer through respective channels.

A method of use of an apparatus as disclosed herein could involve initiating vaporization of the vaporization substance to produce a vapor; and inhaling the vapor through the insulated channel.

Another method of use of an apparatus as disclosed herein could involve initiating vaporization of the vaporization substance to produce a vapor; initiating the auxiliary heater; and inhaling the vapor.

Other aspects and features of embodiments of the present disclosure will become apparent to those ordinarily skilled in the art upon review of the following description.

### BRIEF DESCRIPTION OF THE DRAWINGS

For a more complete understanding of the present disclosure, reference is now made to the following description taken in conjunction with the accompanying drawings, in which:

FIG. 1 is a plan view of an example vaporization device;

FIG. 2 is an isometric view of the vaporization device in FIG. 1;

FIG. 3 is a block diagram of an example vaporization device;

FIG. 4 is a plan and partially exploded view of another example vaporization device;

FIG. 5 is a top view of the chamber in FIG. 4, with a thermal insulator installed;

FIG. 6 is a cross-sectional view of the chamber in FIG. 5, along the line A-A in FIG. 5;

FIG. 7 is a cross-sectional view of another example chamber;

FIG. 8 is a top view of a further example chamber, with a vapor heater;

FIG. 9 is a cross-sectional view of the chamber in FIG. 8, along the line B-B in FIG. 8;

FIG. 10 is an isometric and partially exploded view of another example vaporization device;

FIG. 11 is an isometric and partially exploded view of another example chamber with a cooler;

FIG. 12 is a plan view of an example cap;

FIG. 13 is a plan view of another example cartridge;

FIG. 14 is a cross-sectional and partially exploded view of an example of engagement structures in a vaporization device;

FIG. 15 is a flow diagram illustrating a method according to an embodiment;

FIG. 16 is a flow diagram illustrating a method according to another embodiment;

FIG. 17 is a flow diagram illustrating a method according to a further embodiment; and

FIG. 18 is a flow diagram illustrating a method according to yet another embodiment.

### DETAILED DESCRIPTION

Vaporization devices can be prone to residue buildup from vaporization substances and/or vapor generated from such substances. For example, liquid vaporization substances could leak from a chamber or otherwise leave a residue on a regulator such as a wick and/or elsewhere in a vaporization device. Vapor such as vaporized cannabis resin could also or instead create residue, by condensing or otherwise depositing on vaporization device components as the vapor cools for example. Vaporization substance residue or vapor residue could be problematic, and affect vaporization device operation. Residue could clog a regulator and affect the supply of a vaporization substance to an atomizer for vaporization, for example. Residue could also or instead foul an atomizer and affect vaporization efficiency. Effect, flavor, and/or other characteristics of a vapor could also or instead be affected by residue in a vaporization device.

According to embodiments disclosed herein, temperature is used to prevent and/or reduce residue. Maintaining vapor temperature or re-heating vapor to a higher temperature, for example, could be useful in preventing or reducing at least vapor condensation residue. Components of a vaporization device could also or instead be heated, to potentially help reduce or prevent vaporization substance residue and/or vapor residue buildup.

For illustrative purposes, specific example embodiments will be explained in greater detail below in conjunction with the figures. It should be appreciated, however, that the present disclosure provides many applicable concepts that can be embodied in any of a wide variety of contexts. The specific embodiments discussed are merely illustrative and do not limit the scope of the present disclosure. For example, relative to embodiments shown in the drawings and/or referenced herein, other embodiments may include additional, different, and/or fewer features. The figures are also not necessarily drawn to scale.

The present disclosure relates, in part, to vaporization apparatus such as vaporization devices for vaporization substances that include substances such as cannabinoids or nicotine. However, the vaporization devices described herein could also or instead be used for other types of vaporization substances.

As used herein, the term “cannabinoid” is generally understood to include any chemical compound that acts upon a cannabinoid receptor. Cannabinoids could include endocannabinoids (produced naturally by humans and animals), phytocannabinoids (found in cannabis and some other plants), and synthetic cannabinoids (manufactured artificially).

For the purpose of this specification, the expression “cannabinoid” means a compound such as tetrahydrocannabinol (THC), cannabidiol (CBD), cannabigerolic acid (CBGA), cannabigerol (CBG), cannabigerol monomethylether (CBGM), cannabigerovarin (CBGV), cannabichromene (CBC), cannabichromevarin (CBCV), cannabidiol monomethylether (CBDM), cannabidiol-C4 (CBD-C4), cannabidivarin (CBDV), cannabidiorcol (CBD-C1), delta-9-tetrahydrocannabinol (Δ9-THC), delta-9-tetrahydrocannabinolic acid A (THCA-A), delta-9-tetrahydrocannabionolic acid B (THCA-B), delta-9-tetrahydrocannabinolic acid-C4 (THCA-C4), delta-9-tetrahydrocannabinol-C4, delta-9-tetrahydrocannabivarin (THCV), delta-9-tetrahydrocannabiorcol (THC-C1), delta-7-cis-iso tetrahydrocannabivarin, delta-8-tetrahydrocannabinol (Δ8-THC), cannabicyclol (CBL), cannabicyclovarin (CBLV), cannabielsoin (CBE), cannabinol (CBN), cannabinol methylether (CBNM), cannabinol-C4 (CBN-C4), cannabivarin (CBV), cannabinol-C2 (CBN-C2), cannabiorcol (CBN-C1), cannabinodiol (CBND), cannabinodivarin (CBVD), cannabitriol (CBT), 10-ethoxy-9hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabionol (OTHC), delta-9-cis-tetrahydrocannabinol (cis-THC), 3,4,5,6-tetrahydro-7-hydroxy-alpha-alpha-2-trimethyl-9-n-propyl-2, 6-methano-2H-1-benzoxocin-5-methanol (OH-iso-HHCV), cannabiripsol (CBR), trihydroxy-delta-9-tetrahydrocannabinol (triOH-THC), cannabinol propyl variant (CBNV), and derivatives thereof.

Examples of synthetic cannabinoids include, but are not limited to, naphthoylindoles, naphthylmethylindoles, naphthoylpyrroles, naphthylmethylindenes, phenylacetylindoles, cyclohexylphenols, tetramethylcyclopropylindoles, adamantoylindoles, indazole carboxamides, and quinolinyl esters.

In some embodiments, the cannabinoid is CBD. For the purpose of this specification, the expressions “cannabidiol” or “CBD” are generally understood to refer to one or more of the following compounds, and, unless a particular other stereoisomer or stereoisomers are specified, includes the compound “Δ2-cannabidiol.” These compounds are:
(1) Δ5-cannabidiol (2-(6-isopropenyl-3-methyl-5-cyclohexen-1-yl)-5-pentyl-1,3-benzenediol);
(2) Δ4-cannabidiol (2-(6-isopropenyl-3-methyl-4-cyclohexen-1-yl)-5-pentyl-1,3-benzenediol);
(3) Δ3-cannabidiol (2-(6-isopropenyl-3-methyl-3-cyclohexen-1-yl)-5-pentyl-1,3-benzenediol);
(4) Δ3, 7-cannabidiol (2-(6-isopropenyl-3-methylenecyclohex-1-yl)-5-pentyl-1,3-benzenediol);
(5) Δ2-cannabidiol (2-(6-isopropenyl-3-methyl-2-cyclohexen-1-yl)-5-pentyl-1,3-benzenediol);
(6) Δ1-cannabidiol (2-(6-isopropenyl-3-methyl-1-cyclohexen-1-yl)-5-pentyl-1,3-benzenediol); and (7) Δ6-cannabidiol (2-(6-isopropenyl-3-methyl-6-cyclohexen-1-yl)-5-pentyl-1,3-benzenediol).

In some embodiments, the cannabinoid is THC. THC is only psychoactive in its decarboxylated state. The carboxylic acid form (THCA) is non-psychoactive. Delta-9-tetrahydrocannabinol (Δ9-THC) and delta-8-tetrahydrocannabinol (Δ8-THC) produce the effects associated with cannabis by binding to the CB1 cannabinoid receptors in the brain.

A cannabinoid may be in an acid form or a non-acid form, the latter also being referred to as the decarboxylated form since the non-acid form can be generated by decarboxylating the acid form. Within the context of the present disclosure, where reference is made to a particular cannabinoid, the cannabinoid can be in its acid or non-acid form, or be a mixture of both acid and non-acid forms.

A vaporization substance may include a cannabinoid in its pure or isolated form or in a source material that includes the cannabinoid. The following are non-limiting examples of source materials that include cannabinoids: cannabis or hemp plant material (e.g., flowers, seeds, trichomes, and kief), milled cannabis or hemp plant material, extracts obtained from cannabis or hemp plant material (e.g., resins, waxes and concentrates), and distilled extracts or kief. In some embodiments, pure or isolated cannabinoids and/or source materials that include cannabinoids are combined with water, lipids, hydrocarbons (e.g., butane), ethanol, acetone, isopropanol, or mixtures thereof.

In some embodiments, the cannabinoid is a mixture of THC and CBD. The w/w ratio of THC to CBD in the vaporization substance may be about 1:1000, about 1:900, about 1:800, about 1:700, about 1:600, about 1:500, about 1:400, about 1:300, about 1:250, about 1:200, about 1:150, about 1:100, about 1:90, about 1:80, about 1:70, about 1:60, about 1:50, about 1:45, about 1:40, about 1:35, about 1:30, about 1:29, about 1:28, about 1:27, about 1:26, about 1:25, about 1:24, about 1:23, about 1:22, about 1:21, about 1:20, about 1:19, about 1:18, about 1:17, about 1:16, about 1:15, about 1:14, about 1:13, about 1:12, about 1:11, about 1:10, about 1:9, about 1:8, about 1:7, about 1:6, about 1:5, about 1:4.5, about 1:4, about 1:3.5, about 1:3, about 1:2.9, about 1:2.8, about 1:2.7, about 1:2.6, about 1:2.5, about 1:2.4, about 1:2.3, about 1:2.2, about 1:2.1, about 1:2, about 1:1.9, about 1:1.8, about 1:1.7, about 1:1.6, about 1:1.5, about 1:1.4, about 1:1.3, about 1:1.2, about 1:1.1, about 1:1, about 1.1:1, about 1.2:1, about 1.3:1, about 1.4:1, about 1.5:1, about 1.6:1, about 1.7:1, about 1.8:1, about 1.9:1, about 2:1, about 2.1:1, about 2.2:1, about 2.3:1, about 2.4:1, about 2.5:1, about 2.6:1, about 2.7:1, about 2.8:1, about 2.9:1, about 3:1, about 3.5:1, about 4:1, about 4.5:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 35:1, about 40:1, about 45:1, about 50:1, about 60:1, about 70:1, about 80:1, about 90:1, about 100:1, about 150:1, about 200:1, about 250:1, about 300:1, about 400:1, about 500:1, about 600:1, about 700:1, about 800:1, about 900:1, or about 1000:1.

In some embodiments, a vaporization substance may include products of cannabinoid metabolism, including 11-hydroxy-Δ9-tetrahydrocannabinol (11-OH-THC).

These particulars of cannabinoids are intended solely for illustrative purposes. Other embodiments are also contemplated.

As used herein, the term “terpene” (or “decarboxylated terpene”, which is known as a terpenoid) is generally understood to include any organic compound derived, biosynthetically for example, from units of isoprene. Terpenes may be classified in any of various ways, such as by their sizes. For example, suitable terpenes may include monoterpenes, sesquiterpenes, or triterpenes. At least some terpenes are expected to interact with, and potentiate the activity of, cannabinoids. Examples of terpenes known to be extractable from cannabis include aromadendrene, bergamottin, bergamotol, bisabolene, borneol, 4-3-carene, caryophyllene, cineole/eucalyptol, p-cymene, dihydroj asmone, elemene, farnesene, fenchol, geranylacetate, guaiol, humulene, isopulegol, limonene, linalool, menthone, menthol, menthofuran, myrcene, nerylacetate, neomenthylacetate, ocimene, perillylalcohol, phellandrene, pinene, pulegone, sabinene, terpinene, terpineol, 4-terpineol, terpinolene, and derivatives thereof.

Additional examples of terpenes include nerolidol, phytol, geraniol, alpha-bisabolol, thymol, genipin, astragaloside, asiaticoside, camphene, beta-amyrin, thujone, citronellol, 1,8-cineole, cycloartenol, and derivatives thereof. Further examples of terpenes are discussed in US Patent Application Pub. No. US2016/0250270.

In general, a vaporization substance includes one or more target compounds or components. A target compound or component need not necessarily have a psychoactive effect. One or more flavorants, such as any one or more of: terpene(s), essential oil(s), and volatile plant extract(s), may also or instead be a target compound for vaporization in order to provide flavor to a vapor flow. A vaporization substance may also or instead include other compounds or components, such as one or more carriers. A carrier oil is one example of a carrier.

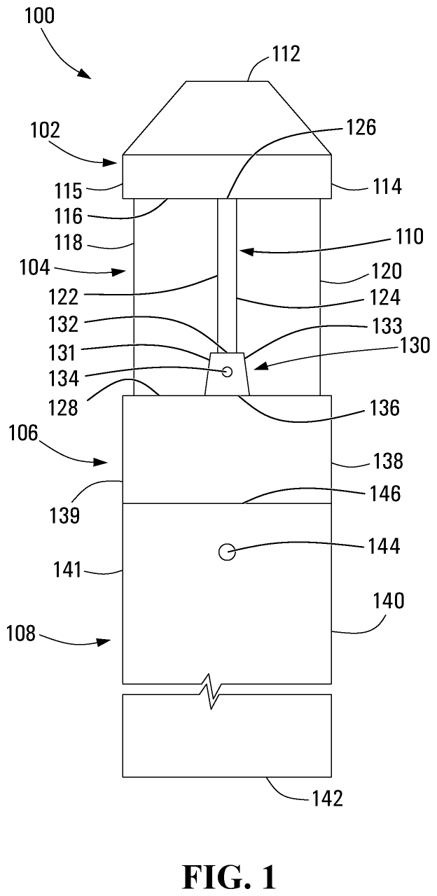

Turning now to vaporization devices in more detail, FIG. 1 is a plan view of an example vaporization device 100. In FIG. 1, the vaporization device 100 is viewed from the side. The vaporization device 100 could also be referred to as a vaporizer, a vaporizer pen, a vape pen or an electronic or “e-” cigarette, for example. The vaporizer 100 includes a cap 102, a chamber 104, a base 106 and a battery compartment 108.

The cap 102 is an example of a lid or cover, and includes a tip 112 and sidewalls 114 and 115, which are sides or parts of the same cylindrical sidewall in some embodiments. The cap 102, in addition to sealing an end of an interior space of the chamber 104, also provides a mouthpiece through which a user can draw vapor from the vaporization device 100 in some embodiments. The mouthpiece is tapered as shown in FIG. 1, and/or otherwise shaped for a user's comfort. The present disclosure is not limited to any particular shape of the cap 102.

The cap 102 could be made from one or more materials including metals, plastics, elastomers and ceramics, for example. However, other materials may also or instead be used.

In other embodiments, a mouthpiece is separate from the cap 102. For example, a cap may be connected to a mouthpiece by a hose or pipe that accommodates flow of vapor from the cap to the mouthpiece. The hose or pipe may be flexible or otherwise permit movement of the mouthpiece relative to the cap, allowing a user to orient the mouthpiece independently from the cap.

The chamber 104 is an example of a vessel to store a vaporization substance prior to vaporization. Although embodiments are described herein primarily in the context of vaporization liquids such as oil concentrates, in general a chamber may store other forms of vaporization substances, including waxes and gels for example. Vaporization substances with water-based carriers are also contemplated. A vaporization device may be capable of vaporizing water-based carriers with emulsified cannabinoids, for example. The chamber 104 may also be referred to as a container, a housing or a tank.

The chamber 104 includes outer walls 118 and 120. Although multiple outer walls are shown in FIG. 1 at 118 and 120, the chamber 104 is perhaps most often cylindrical, with a single outer wall. The outer walls 118 and 120 of the chamber 104 may be made from one or more transparent or translucent materials, such as tempered glass or plastics, in order to enable a user to visibly determine the quantity of vaporization substance in the chamber. The outer walls 118 and 120 are made from one or more opaque materials such as metal alloys, plastics or ceramics in some embodiments, to protect the vaporization substance from degradation by ultraviolet radiation, for example. The outer walls 118 and 120 of the chamber 104 may include markings to aid the user in determining the quantity of vaporization liquid in the chamber. The chamber 104 may have any of a number of different heights and/or other dimensions, to provide different interior volumes.

The chamber 104 engages the cap 102, and may be coupled to the cap, via an engagement or connection at 116. A gasket or other sealing member may be provided between the chamber 104 and the cap 102 to seal the vaporization substance in the chamber.

Some chambers are “non-recloseable” or “disposable” and cannot be opened after initial filling. Such chambers are permanently sealed once closed, and are not designed to be opened and re-sealed. Others are recloseable chambers in which the engagement at 116, between the cap 102 and the chamber 104, is releasable. For example, in some embodiments the cap 102 is a cover that releasably engages the chamber 104 and seals a vaporization substance in the chamber 104. One example of a releasable engagement disclosed elsewhere herein is a threaded engagement or other type of connection, with an abutment between the chamber 104 and the cap 102 but without necessarily an actual connection between the chamber and the cap. Such a releasable engagement permits the cap 102 to be disengaged or removed from the chamber 104 so that the chamber can be cleaned, emptied, and/or filled with a vaporization substance, for example. The cap 102 is then re-engaged with the chamber 104 to seal the vaporization substance inside the chamber.

FIG. 1 also illustrates a stem 110 inside the chamber 104. The stem 110 is a hollow tube or channel through which vapor can be drawn into and through cap 102. The stem 110 may also be referred to as a central column, a central post, a chimney, a hose or a pipe. The stem 110 includes outer walls 122 and 124, although in many embodiments the stem is cylindrical, with a single outer wall. Materials such as stainless steel, other metal alloys, plastics and ceramics may be used for stems such as the stem 110. The stem 110 couples the cap 102 via an engagement or connection 126. Similar to the engagement or connection 116, the engagement or connection 126 is a releasable engagement or connection in some embodiments, and includes a releasable engagement between the stem 110 and the cap 102. In some embodiments, the engagement 126 is in the form of, or includes, a releasable connection.

Although labeled separately in FIG. 1, the engagements at 116 and 126 are operationally related in some embodiments. For example, in some embodiments screwing the cap 102 onto the stem 110 also engages the cap with the chamber 104, or similarly screwing the cap onto the chamber also engages the cap with the stem. This is one example of a threaded connection that also releasably maintains an abutment between the chamber 104 and the cap 102 but without an actual connection between the chamber and the cap.

An atomizer 130 is provided at the base of the stem 110, inside the chamber 104. The atomizer 130 may also be referred to as a heating element, a core, or a ceramic core. The atomizer 130 includes sidewalls 131 and 133, which actually form a single cylindrical or frustoconical wall in some embodiments, and one or more wicking holes or intake holes, one of which is shown at 134. The sidewalls of the atomizer 130 may be made from a metal alloy such as stainless steel, for example. The sidewalls 131 and 133 of the atomizer 130 are made from the same material as the stem 110 in some embodiments, or from different materials in other embodiments.

The atomizer 130 engages, and may couple with, the stem 110 via an engagement 132, and with the base 106 via an engagement 136. Although the engagements 132 and 136 may be releasable, the stem 110, the atomizer 130, and the base 106 are permanently attached together in some embodiments. The atomizer sidewalls 131 and 133 may even be formed with the stem 110 as an integrated single physical component.

In general, the atomizer 130 converts the vaporization substance in the chamber 104 into a vapor, which a user draws from the vaporization device 100 through the stem 110 and the cap 102. Vaporization liquid is drawn into the atomizer 130 through the wicking hole 134 and a wick in some embodiments. The atomizer 130 may include a heating element, such as a resistance coil around a ceramic wick, to perform the conversion of vaporization liquid into vapor. A ceramic atomizer may have an integrated heating element such as a coiled wire inside the ceramic, similar to rebar in concrete, in addition to or instead of being wrapped in a coiled wire. A quartz heater is another type of heater that may be used in an atomizer.

In some embodiments, the combination of the atomizer 130 and the chamber 104 is referred to as a cartomizer.

The base 106 supplies power to the atomizer 130, and may also be referred to as an atomizer base. The base 106 includes sidewalls 138 and 139, which form a single sidewall such as a cylindrical sidewall in some embodiments. The base 106 engages, and may also be coupled to, the chamber 104 via an engagement 128. The engagement 128 is a fixed connection in some embodiments. In other embodiments the engagement 128 is a releasable engagement, and the base 106 can be considered a form of a cover that releasably engages the chamber 104 and seals a vaporization substance in the chamber 104. In such embodiments, the engagement 128 may include a threaded engagement or a threaded connection or an abutment between the chamber 104 and the base 106, for example. A gasket or other sealing member may be provided between the chamber 104 and the base 106 to seal the vaporization substance in the chamber. Such a releasable engagement enables removal or disengagement of the base 106 from the chamber 104 to permit access to the interior of the chamber, so that the chamber can be emptied, cleaned, and/or filled with a vaporization substance, for example. The base 106 is then re-engaged with the chamber 104 to seal the vaporization substance inside the chamber.

The base 106 generally includes circuitry to supply power to the atomizer 130. For example, the base 106 may include electrical contacts that connect to corresponding electrical contacts in the battery compartment 108. The base 106 may further include electrical contacts that connect to corresponding electrical contacts in the atomizer 130. The base 106 may reduce, regulate or otherwise control the power/voltage/current output from the battery compartment 108. However, this functionality may also or instead be provided by the battery compartment 108 itself. The base 106 may be made from one or more materials including metals, plastics, elastomers and ceramics, for example, to carry or otherwise support other base components such as contacts and/or circuitry. However, other materials may also or instead be used.

The combination of a cap 102, a chamber 104, a stem 110, an atomizer 130, and a base 106 is often referred to as a cartridge or “cart”.

The battery compartment 108 could also be referred to as a battery housing. The battery compartment 108 includes sidewalls 140 and 141, a bottom 142 and a button 144. The sidewalls 140 and 141, as noted above for other sidewalls, form a single wall such as a cylindrical sidewall in some embodiments. The battery compartment 108 engages, and may also couple to, the base 106 via an engagement 146. The engagement 146 is a releasable engagement in some embodiments, such as a threaded connection or a magnetic connection, to provide access to the inside of the battery compartment 108. The battery compartment 108 may include single-use batteries or rechargeable batteries such as lithium-ion batteries. A releasable engagement 146 enables replacement of single-use batteries and/or removal of rechargeable batteries for charging, for example. In some embodiments, rechargeable batteries are recharged by an internal battery charger in the battery compartment 108 without removing them from the vaporization device 100. A charging port (not shown) may be provided in the bottom 142 or a sidewall 140, 141, for example. The battery compartment 108 may be made from the same material(s) as the base 106 or from one or more different materials.

The button 144 is one example of a user input device, which may be implemented in any of various ways. Examples include a physical or mechanical button or switch such as a push button. A touch sensitive element such as a capacitive touch sensor may also or instead be used. A user input device need not necessarily require movement of a physical or mechanical element.

Although shown in FIG. 1 as a closed or flush engagement, the engagement 146 between the base 106 and the battery compartment 108 need not necessarily be entirely closed. A gap between outer walls of the base 106 and the battery compartment 108 at the engagement 146, for example, may provide an air intake path to one or more air holes or apertures in the base that are in fluid communication with the interior of the stem 110. An air intake path may also or instead be provided in other ways, such as through one or more apertures in a sidewall 138, 139, elsewhere in the base 106, and/or in the battery compartment 108. When a user draws on a mouthpiece, air is pulled into the air intake path and through a channel. In FIG. 1, the channel runs through the atomizer 130, where air mixes with vapor formed by the atomizer, and the stem 110. The channel also runs through the cap 102 in some embodiments.

The battery compartment 108 powers the vaporization device 100 and allows powered components of the vaporization device, including at least the atomizer 130, to operate. Other powered components could include, for example, one or more light-emitting diodes (LEDs), speakers and/or other elements to provide indicators of, for example, device power status (on/off), device usage status (on when a user is drawing vapor), etc. In some embodiments, speakers and/or other elements generate audible indicators such as long, short or intermittent “beep” sounds as a form of indicator of different conditions. Haptic feedback could also or instead be used to provide status or condition indicators. Varying vibrations and/or pulses, for example, may indicate different statuses or actions in a vaporization device, such as on/off, currently vaporizing, power source connected, etc. Haptic feedback may be provided using small electric motors as in devices such as mobile phones, other electrical and/or mechanical means, or even magnetic means such as one or more controlled electronic magnets.

As noted above, in some embodiments, the cap 102, the chamber 104, the stem 110, the atomizer 130, the base 106 and/or the battery compartment 108 are cylindrical in shape or otherwise shaped in a way such that sidewalls that are separately labeled in FIG. 1 are formed by a single sidewall. In these embodiments, the sidewalls 114 and 115 represent sides of the same sidewall. Similar comments apply to outer walls 118 and 120, sidewalls 131 and 133, outer walls 122 and 124, sidewalls 138 and 139, sidewalls 140 and 141, and other walls that are shown in other drawings and/or described herein. However, in general, caps, chambers, stems, atomizers, bases and/or battery compartments that are not cylindrical in shape are also contemplated. For example, these components may be rectangular, triangular, or otherwise shaped.

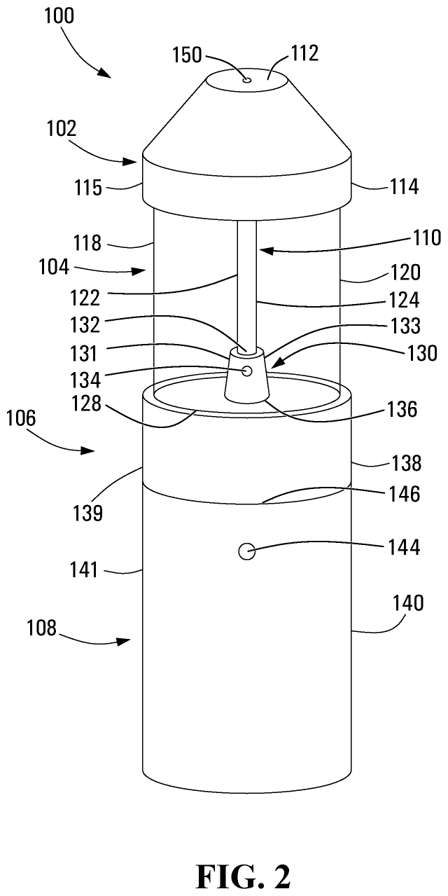

FIG. 2 is an isometric view of the vaporization device 100. In FIG. 2, the cap 102, the chamber 104, the stem 110, the atomizer 130, the base 106 and the battery compartment 108 are illustrated as being cylindrical in shape. As noted above, this is not necessarily the case in other vaporization devices. FIG. 2 also illustrates a hole 150 through the tip 112 in the cap 102. The hole 150 is coupled to the stem 110 through a channel in the cap 102. The hole 150 allows a user to draw vapor through the cap 102. In some embodiments, a user operates the button 144 to vaporize a vaporization substance for inhalation through the cap 102. Other vaporization devices are automatically activated, to supply power from the battery compartment 108 to powered components of the vaporization device when a user inhales through the hole 150. In such embodiments, a button 144 need not be operated to use a vaporization device, and need not necessarily even be provided at all.

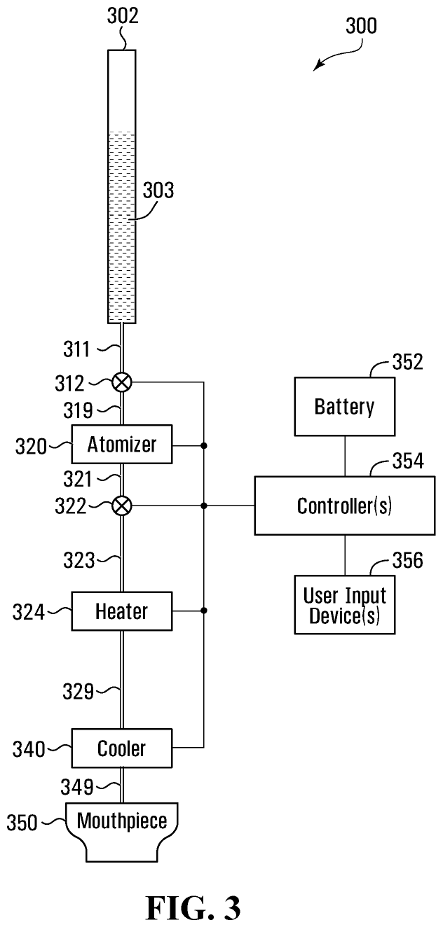

FIG. 3 is a block diagram of an example vaporization device 300. The vaporization device 300 includes a chamber 302 to store a vaporization substance 303. The chamber 302 could be similar to the chamber 104 described above with reference to FIGS. 1 and 2. The chamber 302 could include an engagement structure to engage with a complementary engagement structure of the example device 300. These engagement structures could limit the example device 300 to certain types of chambers, and examples of such engagement structures are disclosed elsewhere herein.

The chamber 302 could be recloseable or non-recloseable. Examples of releasable engagements for recloseable chambers and non-releasable engagements for non-recloseable chambers are provided elsewhere herein.

An atomizer 320 is in fluid communication with the chamber 302 through channels 311, 319 and a regulator in the form of a valve 312 in the example shown, to generate vapor from the vaporization substance 303 by heating the vaporization substance. The valve 312 is an example of a regulator to control movement of the vaporization substance 303 from the chamber 302. Other forms of regulators include, for example, wicks, pumps, mechanical feed structures such as screw conveyors, spray nozzles to spray vaporization substances into the atomizer 320.

Regardless of the types of regulator, a regulator may be useful in providing a measure of dosage control. Dosage of an active ingredient in the vaporization substance 303, for example, could be controlled by controlling the valve 312.

The valve 312 is in fluid communication with the atomizer 320 through channel 319. In some embodiments, the valve 312 could be integrated with the atomizer 320 in a single component. The valve 312 controls the movement of the vaporization substance 303 to the atomizer 320, which generates a vapor by heating the vaporization substance. The atomizer 320 includes a heater to heat the vaporization substance. The heater could include, for example, a coil heater, a fan heater, a ceramic heater, and/or a quartz heater. The atomizer 320 could be implemented as described above with reference to FIGS. 1 and 2.

The vapor produced by the atomizer 320 is fed into a channel 321. The channel 321 is in fluid communication with the atomizer 320, to carry the vapor away from the atomizer. The vapor valve 322 is an example of a vapor regulator, which is provided to control a flow of the vapor from the atomizer.

Various channels such as the channel 321 enable fluid flow through a vaporization apparatus such as a vaporization device, or at least parts thereof. Such fluid may include air, at an intake side of an atomizer for example, or mixture of air and vapor upstream of an atomizer when the atomizer is operating to vaporize a vaporization substance. Fluid flow channels may also be referred to as air channels, but are referenced herein primarily as channels.

The vaporization device 300 also includes an auxiliary heater 324, separate from any heater in the atomizer. The auxiliary heater 324 could include, for example, a coil heater, a fan heater, a ceramic heater, and/or a quartz heater.

The auxiliary heater 324 could be or include a vapor heater, in fluid communication with the atomizer 320 through channels 321, 323 and a vapor regulator in the form of a valve 322 in the embodiment shown, to heat the vapor that is generated by the atomizer. A vapor heater could be at least partially inside a channel such as the channel 323 to heat vapor directly, or outside a channel or air flow path to indirectly heat the vapor. The auxiliary heater 324 could also or instead include a component heater to heat one or more components of the vaporization device 300. More generally, one or more heaters could be implemented to heat the vapor that is generated by the atomizer 320, and/or to heat one or more device components. Heated device components could include, for example, a channel such as 323 that is in fluid communication with the atomizer 320 to carry the vapor away from the atomizer, and/or a regulator such as the valve 312 to control movement of the vaporization substance 303 from the chamber 302 to the atomizer.

A cooler 340 is provided in some embodiments, to cool the vapor. The cooler 340 could be in fluid communication with the atomizer 320, with the heater 324, or with a channel that is in fluid communication with the atomizer and/or with the heater. In the example shown, the cooler 340 is in fluid communication with the heater 324 through a channel 329, which could include one or more vapor regulators. The cooler 340 could be at least partially inside a channel such as the channel 329 to cool vapor directly, or outside a channel or fluid flow path to indirectly cool the vapor by cooling one or more components through which the vapor flows.

The mouthpiece 350 is in fluid communication with the atomizer 320, with the heater 324, with the cooler 340, and with the channels 321, 323, 329 therebetween. In general, the mouthpiece 350 could be directly or indirectly in fluid communication with other components. The channel 349, for example, could be a hose or other channel through which the mouthpiece 350 is indirectly in fluid communication with other components of the vaporization device 300. Like other channels in FIG. 3, the channel 349 could include one or more vapor regulators.

In some embodiments, the channel 349 and/or the mouthpiece 350 could also or instead provide vapor cooling. Characteristics such as length and/or material composition of the channel 349, which could be or include a mouthpiece hose for example, could be selected to provide for cooling of vapor. A longer channel 349 provides more time for vapor to cool before reaching the mouthpiece 350 and being inhaled by a user. A channel 349 that is made from or at least includes one or more thermally conductive materials could provide or improve vapor cooling prior to inhalation. Cooling could also or instead be provided by one or more additional air intakes, in the mouthpiece 350 and/or elsewhere in a vaporization device, to admit air into a vapor flow to cool the vapor.

The valve 312, the atomizer 320, the vapor valve 322, the auxiliary heater 324, and the cooler 340 are controlled by one or more controllers 354. A controller at 354 could be implemented, for example, using hardware, firmware, one or more components that execute software stored in one or more non-transitory memory devices (not shown), such as a solid-state memory device or a memory device that uses movable and/or even removable storage media. Microprocessors, Application Specific Integrated Circuits (ASICs), Field Programmable Gate Arrays (FPGAs), and Programmable Logic Devices (PLDs) are examples of processing devices that could be used to execute software.

A power source such as a battery 352 and one or more user input devices 356 are coupled to the controller(s) 354. The user input device(s) 356 could include switches, sliders, dials, and/or other types of input device that enable a user to control any of various aspects or parameters of the valve 312, the atomizer 320, the vapor valve 322, the auxiliary heater 324, and/or the cooler 340. Other input device examples are disclosed elsewhere herein, with reference to the button 144 in FIGS. 1 and 2, for instance.

The battery 352 provides power to the controller(s) 354, which could then provide power to other components of the example device 300. The valve 312 and/or the vapor valve 322 could be controlled in this type of implementation by controlling power to the valve. For example, the valve 312 and/or the vapor valve 322 could be normally closed when not supplied with power, and opened when powered. In other embodiments, power and control are implemented separately. Other control mechanisms are also possible. However, not all types of regulators are necessarily controlled. A wick, for example, draws a vaporization substance from a chamber to an atomizer for vaporization, but the wick itself is not controlled.

A controller at 354 also controls and supplies power to the atomizer 320, and could provide on-off power control based on operation of a power button or switch at 356 or a user inhaling on the device 300, for example. In some embodiments, different voltages and/or currents could be supplied to the atomizer 320 to enable the atomizer to provide different temperatures for vaporization. This type of power control, which could be considered a form of temperature control, could be provided through a user input device 356, and/or based on sensing the type of chamber 302 currently installed in the device 300. For example, the chamber 302 could include an indicator of its vaporization substance 303. Using this indicator, a controller 354 could determine a vaporization temperature that is appropriate for the vaporization substance 303, and control the power delivered to the atomizer 320 accordingly. The voltage, current, and/or power supplied to the atomizer 320 could also or instead be controlled based on a desired flow or quantity of vapor produced by the atomizer, which could be selected or otherwise controlled using a user input device 356, for example.

The same controller or a different controller at 354 could control and power the heater 324. This control could be similar to the control of the atomizer 320 discussed above. In some embodiments, different voltages and/or currents could be supplied to the heater 324 to heat the vapor produced by the atomizer 320 and/or to heat one or more other components of the vaporization device 300 to any of various temperatures. These temperatures could be set by a user input device 356, and/or determined based on such parameters as any one or more of: the type of vaporization substance 303, the vaporization temperature used by the atomizer 320, vapor temperature at one or more measuring or sensing points along a channel, length of a channel, composition of a channel, intake air temperature, and desired vapor output temperature at the mouthpiece 350. For example, as noted above, the chamber 302 could include an indicator of its vaporization substance 503. Power to the heater 324 could be controlled based on one or more of these indicators. A controller 354 could control the heater 324 to heat the vapor produced by the atomizer 320 to a temperature that is expected to prevent or reduce vapor residue, by condensation for example. Power supplied to the heater 324 could be turned off, or the heater could be otherwise disabled, if the vapor temperature produced by the atomizer 320 is sufficient to prevent or reduce vapor residue.

Some components of the vaporization device 300 could be easier to clean, and/or be less affected by residue, than other components. For example, it could be much easier for a user to detach and clean the mouthpiece 350 than the atomizer 320 or the heater 324. Vapor heating temperatures could be determined accordingly, and set to a temperature that is expected to prevent or reduce residue buildup within at least a certain distance along a channel or at least at certain components of a vaporization device. Residue buildup might be less problematic in the mouthpiece 350, for example, and vapor heating temperatures could be set to help prevent or reduce residue buildup at least upstream from the mouthpiece.

In some embodiments, target temperatures for vapor heating, and/or for maintaining vapor temperature, are in the range of 150° C. to 180° C. Different vaporization substances or ingredients therein could have different vaporization temperatures, and target temperatures could be determined based on the vaporization temperature(s) of the particular vaporization substance(s) or ingredient(s) that are to be vaporized. Waxes, for example, tend to be very temperature sensitive and to prevent buildup of waxes a vaporization device could be run at higher temperatures, at about 195° C. in an embodiment. Temperatures at which vapor may condense or deposit could be different from vaporization temperatures, and target temperatures could also or instead be determined based on condensation and/or deposition temperature(s). One or more target temperatures could be used not only in vapor heating embodiments, but also or instead in other embodiments disclosed herein, such as embodiments in which one or more vaporization device elements are insulated and/or one or more one or more vaporization device elements are heated.

With an auxiliary heater 324, vapor temperature at the mouthpiece 350 would be expected to be higher than for a vaporization device without auxiliary heating. A controller at 354, which could be the same controller that controls other components or a different controller, could control and power the cooler 340 to reduce vapor temperature. This control could be similar to the control of the atomizer 320 and/or the heater 324 discussed above. In some embodiments, different voltages and/or currents could be supplied to the cooler 340 to cool the vapor produced by the atomizer 320 and/or to cool one or more other components of the vaporization device 300 to any of various temperatures.

Cooling temperatures could be set by a user input device 356, and/or determined based on such parameters as any one or more of: the type of vaporization substance 303, the vaporization temperature used by the atomizer 320, vapor temperature at one or more measuring or sensing points along a channel, length of a channel, composition of a channel, intake air temperature, and desired vapor output temperature at the mouthpiece 350. For example, power to the cooler 340 could be controlled based on a temperature reading by one or more temperature sensors. Incoming vapor temperature in the channel 329 and/or outgoing vapor temperature in the channel 349, for example, could be sensed and used by a controller 354 to turn the cooler 340 on or off, and/or to control a cooling temperature of the cooler 340. Power supplied to the cooler 340 could be turned off, or the cooler could be otherwise disabled, if a sensed vapor temperature at a desired temperature or within a desired temperature range.

As noted above, some components of the vaporization device 300 could be easier to clean, and/or be less affected by residue, than other components. The cooler 340 could be positioned at or upstream of, or potentially integrated with, such easier cleaned or less affected components. Vapor heating could therefore be effective for components that are upstream of the cooler 340, but not significantly affect components that are downstream of the cooler in a direction of vapor flow.

A specific example of a vaporization device 300 is shown in FIG. 3. Other embodiments are also contemplated.

For example, multiple chambers to store respective vaporization substances could be provided. A chamber could be in fluid communication with a respective atomizer, multiple chambers could supply their respective vaporization substances to the same atomizer, and/or one or more chambers could supply their vaporization substance(s) to a channel or other component and not to an atomizer. Multiple channels, in fluid communication with different atomizers, chambers, or air intakes for example, could be provided.

Either or both of the valve 312 and the vapor valve 322 could be excluded in other vaporization devices. Valves or vapor valves could also or instead be provided in different channels.

Embodiments could include more than one auxiliary heater 324. Some embodiments include no auxiliary heater. An insulated channel, for example, could help maintain a higher temperature of a vapor that is generated by the atomizer 340 and potentially prevent or reduce residue buildup. An insulated channel could include any or all of the channels 321, 323, 329 in FIG. 3, and even the channel 349 if no cooler 340 is provided for example.

Although the channels 321, 323, 329, and 349 are all illustrated separately, these channels could instead form a single continuous channel from the atomizer 320 to the mouthpiece 350. At least a portion of the vapor valve 322, the heater 324, and/or the cooler 340 could be inside of this continuous channel.

The vaporization substance 303 could be in the form of a dry substance, liquid, gel and/or a wax, and could have any of various effects. For example, some vaporization substances could include one or more active ingredients that have a psychoactive effect, whereas others could include flavorants such as any one or more of: terpenes, an essential oil, and a volatile plant extract. In a multi-chamber embodiment, one or more vaporization substances could contain an active substance, and others could include flavorants. A user could, using one or more user input devices 356, selectively vaporize the active substance(s) and flavorant(s) to create a controllable mixture of vapors produced from the vaporization substances. This mixture could be tuned for a specific effect, flavor and/or aromatic profile desired the by the user.

FIG. 3 illustrates a general example of a vaporization device with multiple chambers in a serial configuration. Specific examples of vaporization devices with residue prevention or reduction will now be discussed.

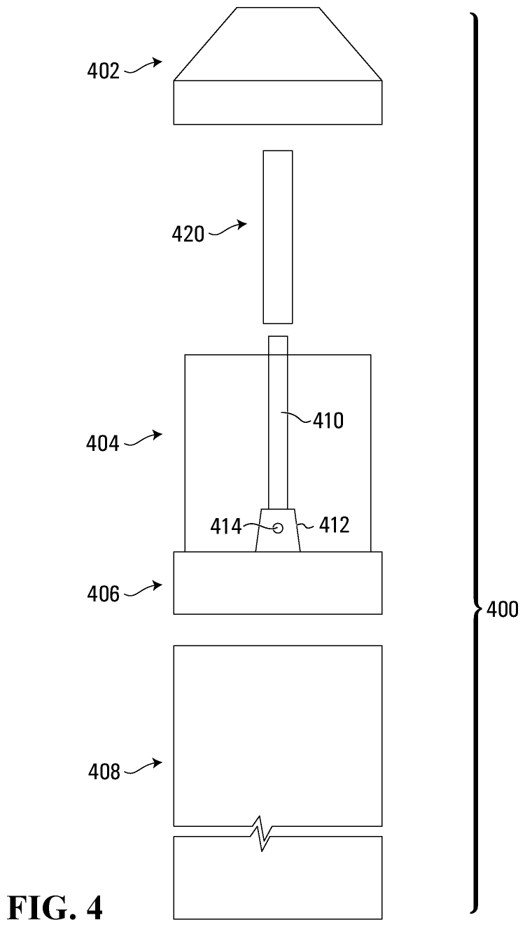

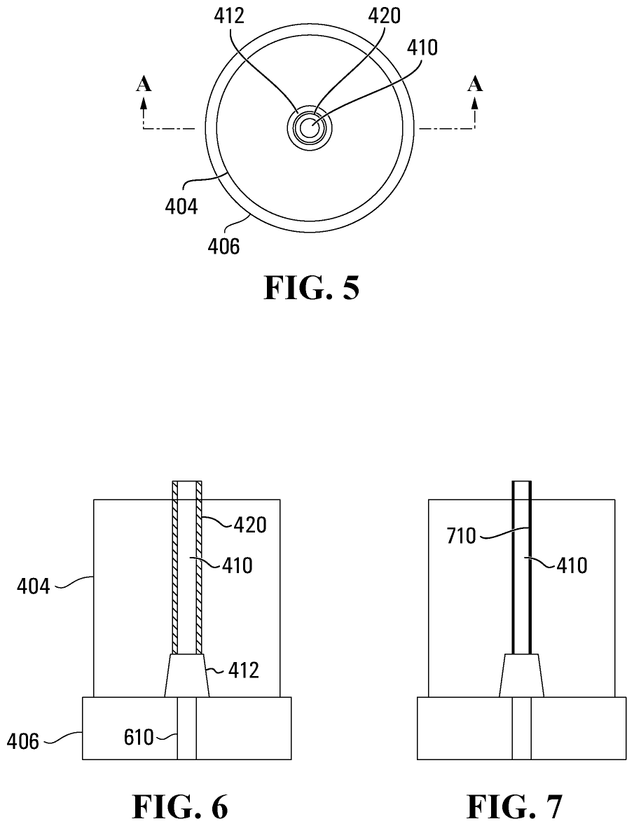

FIG. 4 is a plan and partially exploded view of another example vaporization device 400, FIG. 5 is a top view of the chamber 404 in FIG. 4, with a thermal insulator 420 installed, and FIG. 6 is a cross-sectional view of the chamber in FIG. 5, along the line A-A in FIG. 5. Various features referenced in the description below are shown in one or more of these drawings.

The vaporization device 400 includes, in part, a cap 402, a chamber 404, a base 406, and a battery compartment 408. A stem 410, an atomizer 412, and an intake hole 414 are also shown inside the chamber 404. These components could be similar to the cap 102, the chamber 104, the base 106, the battery compartment 108, the stem 110, the atomizer 130 and the intake hole 134 discussed above with reference to FIGS. 1 and 2. A thermal insulator 420 is also shown, and in this embodiment the thermal insulator is to insulate the stem 410 along at least part of its length and provide an insulated channel.

The thermal insulator 420 could be made from or include any of various thermally insulative materials that are relatively poor conductors of temperature. Examples of thermally insulative materials include glass, ceramics, and other compositions such as plastic and Calcium Silicate, with any of various internal structures such as cellular glass and fibrous materials.

The thermal insulator 420 could be a tube or sleeve that fits over the stem 410 and could, but need not necessarily in every embodiment, be sealed against the stem 410, the atomizer 412, and/or the cap 402. Sealing elements such as o-rings or gaskets could be used to provide seals.

The thermal insulator 420 itself could be sealed from a vaporization substance in the chamber 404. For example, the thermal insulator 420 could be or include a fibrous or absorbent material that is enclosed within a liquid-proof membrane or otherwise sealed from the vaporization substance so that the thermal insulator can be located at an outside part of the insulated channel, as shown perhaps most clearly in FIGS. 5 and 6, without absorbing or otherwise retaining the vaporization substance.

When assembled, the thermal insulator 420 covers at least part of the stem 410, and the top ends of at least the chamber 404 and the stem 410 engage the cap 402. The chamber 404 and base 406 are illustrated in an assembled state, with the chamber engaging the base. The base 406 also engages the battery compartment 408 when the device 400 is fully assembled. Examples of cap/chamber/stem/base/battery compartment engagements are described elsewhere herein, with reference to FIGS. 1 and 2. The thermal insulator 420 could also engage with the cap 402 in some embodiments.

The chamber 404 could be recloseable or non-recloseable. Examples of releasable engagements for recloseable chambers and non-releasable engagements or non-recloseable chambers are provided elsewhere herein.

The chamber 404 stores a vaporization substance. The atomizer 412 is in fluid communication with the chamber 404, through the intake hole 414, to generate vapor from this vaporization substance by heating the vaporization substance. The stem 410 provides a channel in fluid communication with the atomizer 412, to carry the vapor away from the atomizer. Vapor produced in the atomizer 414 flows through this channel. The channel is in fluid communication with an intake channel 610 (FIG. 6), which is provided in the base 406. The cap 402, which could include a mouthpiece, is also in fluid communication with the channel in the stem 410.

According to the embodiment shown in FIG. 6, the stem 410 is thermally insulated by the thermal insulator 420 along its entire length. More generally, an insulated channel could extend at least partially along the stem 410. The entire stem 410 need not necessarily be insulated. Characteristics such as the type(s) of thermal insulator, the amount of thermal insulation, and/or how much of a channel is thermally insulated could be determined based on any one of more of various parameters. Examples of such parameters include user input, expected vapor temperature exiting the atomizer 612, measured vapor temperature exiting the atomizer, expected vapor temperature drop along the stem 610, measured vapor temperature drop along the stem, and/or vapor condensation temperature, for example.

FIGS. 4 to 6 illustrate an embodiment in which a thermal insulator 420 is located at an outside part of the insulated channel. Other embodiments are also contemplated. FIG. 7, for example, is a cross-sectional view of another example chamber in which a thermal insulator 710 is located at an inside part of the insulated channel, inside the stem 410. The thermal insulator 710 could be implemented as disclosed elsewhere herein, in the same way as the thermal insulator 420 (FIG. 6) for example. An internal thermal insulator 710 need not necessarily be sealed from the vaporization substance in the chamber, because it would be exposed to vapor that is generated from the vaporization substance rather than to the vaporization substance itself. The thermal insulator 710 could, however, still be sealed to provide a smooth surface for vapor flow and/or to prevent or reduce vapor absorption. A chamber with an internal thermal insulator 710 could otherwise be substantially the same as a chamber with an thermal insulator 420.

On a comparison of FIGS. 6 and 7, it will be seen that the external thermal insulator 420 is illustrated as being thicker than the internal thermal insulator 710. This is just an example. A thinner thermal insulator 710 might be preferred as an internal thermal insulator so as to avoid overly restricting the size of the internal channel through the stem 410. Alternatively, channel size could be maintained by using a thicker internal thermal insulator with a larger diameter stem.

Whether internal or external, a thermal insulator could be removable, for replacement or cleaning for example. A thermal insulator could be placed over or inside a stem without being fastened to the stem, and then slid on or into, and off or out of, the stem. Any fasteners could be released or broken, and then re-fastened or replaced, when a thermal insulator is removed and re-installed or replaced.

In some embodiments, an insulated channel also or instead includes a thermal insulator in the form of a coating on the insulated channel. A coating could be inside and/or outside a stem, for example, similar to the embodiments shown in FIGS. 6 and 7.

Other embodiments are also possible. An insulated channel could itself be made from or include a thermally insulative material. A stem could be made from such a material, for example.

A thermally insulative material could otherwise be integrated with an insulated channel. For example, a channel could have multiple walls or otherwise provide one or more internal cavities to improve thermal insulation properties. The internal cavity or cavities could be filled with a thermally insulative material.

Thermally insulating a channel does not in any way preclude thermal insulation of other components. Referring to FIGS. 6 and 7, for example, a thermal insulator 420, 710 could extend lower, to at least partially cover an outside wall of an atomizer. Even an intake hole 414 and/or a regulator could be covered by a thermal insulator that is permeable to the vaporization substance that is to be stored in a chamber. Atomizer and/or regulator components could also or instead be coated with a thermal insulator, made from a thermally insulative material, or otherwise include a thermally insulative material.

In the embodiments in FIGS. 4 to 7, thermal insulation of at least a channel may help prevent or reduce residue deposition by maintaining higher vapor temperature relative to vapor temperature that would be expected without insulation. Another possible residue prevention or reduction technique involves heating vapor and/or one or more vaporization device components.

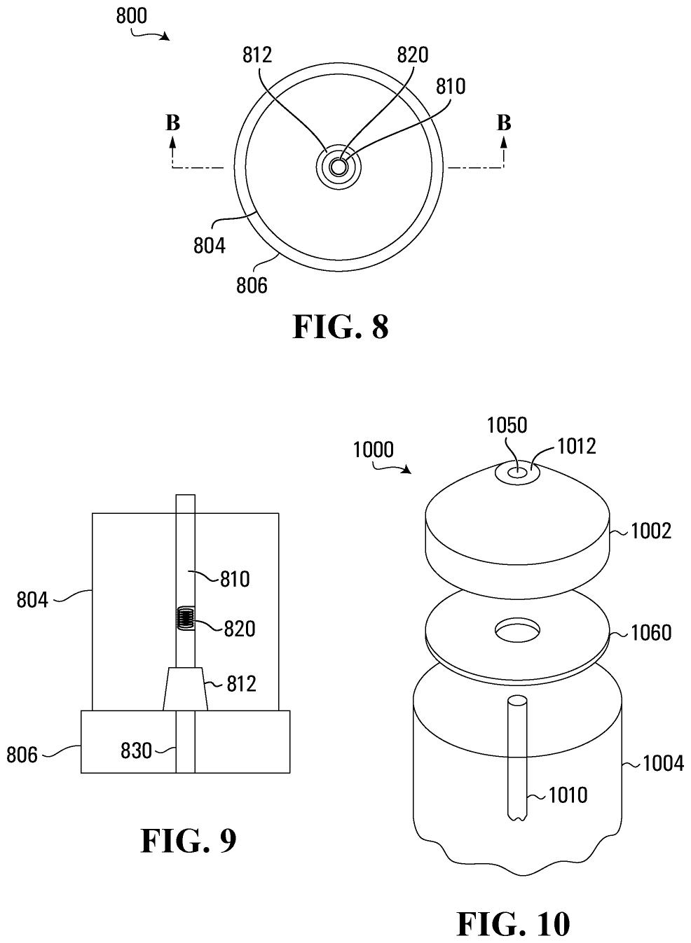

FIG. 8 is a top view of a further example chamber 804 with a vapor heater, and FIG. 9 is a cross-sectional view of the chamber in FIG. 8, along the line B-B in FIG. 8. Features referenced below are shown in one or both of FIGS. 8 and 9.

The chamber 804 engages a base 806. In a vaporization device, the top of the chamber 804 and a stem 810 could engage a cap, and the bottom of the base 806 could engage a battery compartment. In addition to the stem 810, an atomizer 812, and an auxiliary heater 820 are also shown inside the chamber 804. The stem 810 provides a channel that is in fluid communication with a channel 830 in the base. The chamber 804, the base 806, the stem 810, and the atomizer 812 could be similar to the chamber 104, 404, the base 106, 406, the stem 110, 410, and the atomizer 130, 412 and the intake hole 134 discussed above with reference to FIGS. 1, 2, and 4.

The heater 820 is separate from a vaporization heater in the atomizer 812 and in the illustrated embodiment is a vapor heater to heat the vapor that is generated by the atomizer. The heater 820 is illustrated as a coil heater, however a fan heater, a ceramic heater, and/or another type of heater such as a quartz heater could also or instead be used. Various control options for controlling an auxiliary heater such as the heater 820 are disclosed elsewhere herein. The location of the heater 820 along the stem 810 could be determined based one or more parameters such as expected vapor temperature exiting the atomizer 812, measured vapor temperature exiting the atomizer, expected vapor temperature drop along the stem 810, measured vapor temperature drop along the stem, and/or vapor condensation temperature, for example.

At least a heating element of a vapor heater, such as a coil as shown at 820, could be positioned inside a channel. Power and/or control connections could be located inside or outside the channel. In some embodiments, the base 806, the atomizer 812, and the stem 810, or elements therein, act as a conductor to provide a connection that delivers power to the heater 820 from a battery in a battery compartment with which the base engages. However, one or more separate electrical conductors could be provided, for example, from the base 806 and along an inner or outer wall of the stem 810, along an outer or inner wall of the chamber 804, and/or elsewhere in a vaporization device to deliver power to the heater 820. The heater 820 could be electrically coupled to the atomizer 812 or to power and/or control terminals or connections in the atomizer, with internal conductors inside the stem 810 for example. Conductors could be implemented using transparent conductors, such as indium tin oxide films, so that they are not noticeable to a user. Alternatively, a separate power source such as a battery could be provided to power the heater 820.

An auxiliary heater could also or instead include a heater to heat one or more components of a vaporization device. For example, a heater could be provided, inside or outside the stem 810, to heat the stem itself. The heater 820 could, to at least some extent, also heat the stem 810 and thereby help maintain or increase vapor temperature. A component heater could be or include a fan heater, a ceramic heater, and/or another type of heater such as a quartz heater or a radiant heater. In some embodiments, vaporization device components themselves could have integrated heating elements. A heater in the form of a resistance wire or heating coil, for example, could be integrated into the stem 810, at least the outer wall of the atomizer 812, and/or a regulator to control movement of a vaporization substance from the chamber 804 to the atomizer.

Thus, an auxiliary heater could be or include a component heater to heat one or more vaporization device components, such as a channel that is in fluid communication with an atomizer to carry the vapor away from the atomizer, and a regulator to control movement of the vaporization substance from the chamber to the atomizer. At least parts of an atomizer could be heated in some embodiments, although heat generated by the atomizer itself during vaporization could be sufficient to prevent or reduce residue buildup on or in the atomizer.

A vaporization device could include a cooler in some embodiments, to reduce the temperature of the final vapor before inhalation, so that the vapor is pleasurable and safe to inhale. In some embodiments, the cooler is in fluid communication with a channel, to directly cool the vapor. Vapor cooling could also or instead be indirect. A cooler could cool a component through which the vapor flows, for example.

The cooler could be or include an active cooler such as a thermoelectric cooler. A cooler could also or instead include a passive cooler such as a heat sink to absorb heat from the vapor.

FIG. 10 is an isometric and partially exploded view of another example vaporization device 1000, which includes a passive cooler in the form of a heat sink 1060. The example vaporization device 1000 also includes a cap 1002 with a tip 1012 and a hole 1050 through which a user inhales, and a chamber 1004 with a stem 1010. The top of the chamber 1004 and the stem 1010 engage the cap 1002 when the vaporization device 1000 is assembled. The chamber 1004 and the stem 1010 could be the same as those disclosed in other embodiments herein. The vaporization device 1000 could also include other components, such as a base and a battery compartment as in other disclosed embodiments.

The heat sink 1060 could form part of a channel through which vapor flows, and thereby absorb heat from the vapor to reduce temperature of the vapor. The heat sink 1060 could also or instead be positioned around, and possibly be in contact with, part of the stem 1010 to indirectly cool the vapor by absorbing heat from the stem. The heat sink could be made from or at least include a thermally conductive material such as a metal. The location of the heat sink 1060 along the stem 1010 could be determined based one or more parameters such as expected vapor temperature exiting an atomizer that is in fluid communication with the stem, measured vapor temperature exiting the atomizer, expected vapor temperature drop along the stem, measured vapor temperature drop along the stem, and/or vapor condensation temperature, for example.

The vaporization device 1000 could carry the heat sink 1060 in any of various ways. For example, the heat sink 1060 could be integrated with the cap 1002. The cap 1002 could be molded around the heat sink 1060 to encapsulate the heat sink. The heat sink 1060 could instead be coupled to the cap 1002, by adhesive or otherwise. A friction fit engagement between the heat sink 1060 and the cap 1002 could also or instead be used to couple the heat sink to the cap, to have the heat sink carried by inside surfaces of outer cap walls, by one or more structures such as posts at a bottom surface of the cap, or otherwise carried in a cavity in the cap, for example.

The heat sink 1060 need not necessarily be carried by the cap 1002, and could instead be coupled to or integrated with the stem 1010, to the chamber 1004, or to another component. For example, a carrier or adapter could be provided between the cap 1002 and the chamber 1004 and/or the stem 1010 to carry the heat sink 1060, and the heat sink could be coupled to that carrier or adapter.

In some embodiments, the heat sink 1060 could be a removable heat sink element. Such a removable heat sink element could be coupled to the cap 1002 in the embodiment in FIG. 10, or more generally coupled to an apparatus such as a cartridge or a vaporization device, by a releasable coupling. A friction fit engagement with the cap 1002 represents one example of a releasable engagement, and could potentially be applied to a vaporization device component other than a cap. A threaded engagement is another example. A removable heat sink element could also or instead be coupled to a cap or other part of an apparatus such as a cartridge or a vaporization device magnetically.

A removable heat sink element could be removed for cleaning, for example, and then re-installed. A removable heat sink element could also or instead be removed and cooled by refrigeration before use.

Although only one heat sink 1060 is shown in FIG. 10, multiple heat sink elements could be provided, for a higher cooling capacity. Multiple heat sink elements could be releasably coupled to each other, and/or to an apparatus, magnetically or otherwise.

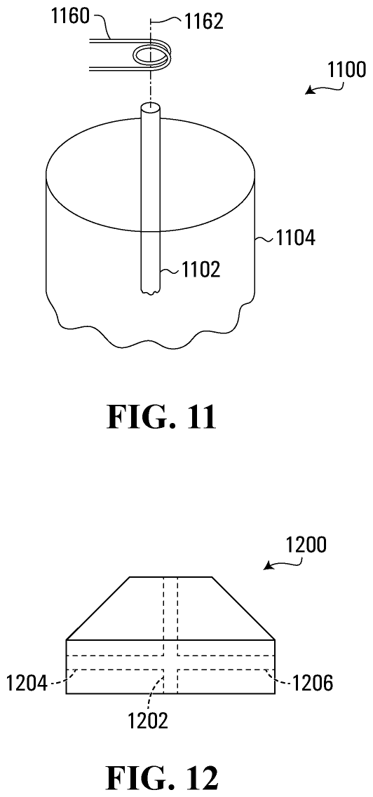

The heat sink 1060 is one example of a cooler. FIG. 11 is an isometric and partially exploded view of another example chamber 1104 with a different type of cooler 1160. The cooler 1160 includes an element with a turn that, when installed, surrounds and could be in contact with part of the stem 1102. This is represented in FIG. 11 by the dashed line 1162. The location of the cooler 1160 along the stem 1102 could be determined based one or more parameters such as expected vapor temperature exiting an atomizer that is in fluid communication with the stem, measured vapor temperature exiting the atomizer, expected vapor temperature drop along the stem 1102, measured vapor temperature drop along the stem, and/or vapor condensation temperature, for example.

The chamber 1104 and the stem 1102 could be the same as those disclosed in other embodiments herein, and could be part of a vaporization device that also includes other components, such as a cap, a base, and a battery compartment as in other disclosed embodiments.

The chamber 1104 and/or another part of an apparatus such as a cartridge or a vaporization device could carry the cooler 1160 in any of various ways. For example, the cooler 1160 could be integrated with the stem 1102 or a cap. A cap could be molded around the cooler 1160 to encapsulate at least part of the cooler. The cooler 1160 could instead be coupled to a cap and/or another part of an apparatus, by adhesive or otherwise. A friction fit engagement between the cooler 1160 and part of an apparatus could also or instead be used to couple the cooler to an apparatus. In some embodiments, the cooler 1160 could be or include a removable cooling element, and be coupled to an apparatus by a releasable coupling, examples of which are disclosed elsewhere herein.

The cooler 1160 could be solid or hollow, and formed from a thermally conductive material such as a metal. In some embodiments, the cooler 1060 is a heat exchanger, to transfer heat from the vapor to a heat sink. The heat sink could be or include a material inside the heat exchanger. For example, the cooler 1060 could be hollow, with a gas such as air or a liquid such as a refrigerant as a heat sink inside. A gas or liquid heat sink could be circulated, by a fan or pump for example, to transfer heat away from the stem 1102. In the case of air as the heat sink, outside air could be circulated through the cooler 1160. A heat sink material could instead be part of a closed, sealed system.

In the examples of a gas or liquid heat sink, the heat sink is inside the cooler 1060 and is thermally coupled to the vapor, indirectly through the stem 1102. In other embodiments, a heat sink could be physically in contact with or otherwise thermally coupled to a heat exchanger without being inside the heat exchanger.

A passive cooler as referenced herein is a cooler that includes no powered or controlled components, whereas an active cooler includes one or more powered and/or controlled components. The heat sink 1060 in FIG. 10 could be a passive cooler, and embodiments of the cooler 1160 in FIG. 11 described herein include active and passive cooler embodiments. A thermoelectric cooler is another example of an active cooler. Various control options for controlling an active cooler are disclosed elsewhere herein.

Power and/or control connections for an active cooler could be located inside or outside the channel. In some embodiments, a base, an atomizer, and a stem, or elements therein, act as a conductor to provide a connection that delivers power to an active cooler from a battery in a battery compartment with which the base engages. However, one or more separate electrical conductors could be provided, for example, from a base and along an inner or outer wall of a stem, along an outer or inner wall of a chamber, and/or elsewhere in a vaporization device to deliver power to an active cooler. An active cooler could be electrically coupled to power and/or control terminals or connections in an atomizer, with internal conductors inside a stem for example. Conductors could be implemented using transparent conductors, such as indium tin oxide films, so that they are not noticeable to a user. Alternatively, a separate power source such as a battery could be provided to power an active cooler.

FIG. 11 illustrates an example of a cooler 1160 that is outside a channel, in particular outside the stem 1102. In some embodiments, a cooler is at least partially inside a channel. At least a cooling element such as a coil of the cooler 1160, for example, could be inside the stem 1102. This could be similar to the arrangement shown in FIG. 9 for the auxiliary heater 820.

Although the cooler 1160 is shown in FIG. 11 with a cooling coil that has only one turn, a cooler could have multiple turns to provide a higher cooling capacity. Multiple separate coils could also or instead be provided.

A cooler or heat exchanger need not be in the form of a coil, and could take other shapes or forms, such as a mesh, grid, plate, ring, or sleeve that is inside or outside a channel or forms part of a channel.

A cap or mouthpiece could also or instead provide a cooling effect. FIG. 12 is a plan view of an example cap 1200, with a channel 1202 for fluid communication with a mouthpiece, which could be part of the cap or a separate component. The cap 1200 includes additional air intake channels 1204, 1206 through which ambient air outside the cap can enter the cap and mix with vapor in the channel 1202 to cool the vapor. Control of intake air flow in the channels 1204, 1206 could be manual and/or automatic. A user could manually control intake air flow by operating one or more valves and/or other air flow control component(s) to provide a desired temperature at an outlet of the channel 1202. Automatic control could be responsive to one or more temperature sensors to sense temperature of air in a channel, such as the channel 1202 and/or an upstream channel in fluid communication with the channel 1202, and provide measurements and/or other signals to control operation of one or more air flow control components. Another intake air control option would be to control one or more air flow control components based on operation of an atomizer and/or an auxiliary heater. For example, an auxiliary heater and one or more intake air flow control components could be operated or controlled together, to increase intake air flow when the auxiliary heater is in operation and to decrease intake air flow when the vapor heater is not in operation.

Cooling by intake air as shown by way of example in FIG. 12 could be implemented in a cap 1200 and/or in a mouthpiece that is in fluid communication with a channel through which vapor flows. A cap or a mouthpiece could be releasably coupled to an apparatus such as a chamber, a cartridge, or a vaporization device, by a threaded engagement, by a friction fit engagement, or by some other type of releasable engagement.

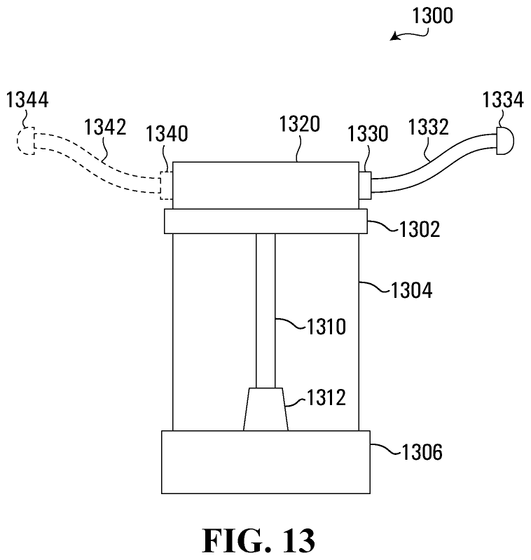

Vapor cooling could also or instead be provided by implementing a longer channel for the vapor to travel through before reaching a mouthpiece. FIG. 13 is a plan view of another example cartridge 1300 that includes such longer channels.

The example cartridge 1300 includes a chamber 1304 with a stem 1310 and an atomizer 1304, and a base 1306 engaged with the chamber 1304. These components could be as disclosed, for example, with reference to FIGS. 1 and 2. A cap 1302 engages the chamber 1304 and the stem 1310, and the engagements of the chamber and the stem with the cap could also be as disclosed, for example, with reference to FIGS. 1 and 2. A mouthpiece 1334 is coupled to the cap through a hose or pipe 1332, a connector 1330, and a manifold 1320 in the example shown, but in other embodiments the hose could be coupled to the cap 1302. Multiple mouthpieces could be provided in some embodiments, and a second mouthpiece 1344, hose 1342, and connector 1340 are shown in FIG. 13.

The manifold 1320 provides multiple channels in fluid communication with the channel through the stem 1310, and could be made from the same material(s) as the cap 1302 and/or different material(s). The manifold 1320 and the cap 1302 could be integrated together into a single component in some embodiments.

The connectors 1330, 1340 could be, for example, threaded connectors to couple the hoses 1332, 1342 to the manifold 1320. Any of various types of connectors, made from the same material(s) as the manifold 1320 and/or different material(s), could be used for this purpose. Threaded connectors, friction fit connectors, magnetic connectors and/or other types of connectors could be used. The manifold 1320 and/or the connectors 1330, 1340 could include valves or other regulators that open channels through the connectors only when a mouthpiece hose 1332, 1342 is connected.

The hoses 1332, 1342 could be made from any of various materials, such as rubber or plastic. Hoses 1332, 1342 that are made from, or at least include, a thermally conductive material, could improve vapor cooling as vapor travels along a hose. For example, the hoses 1332, 1342 could be made from or at least include materials with a high thermal conductivity, such as copper, to help cool the vapor. Each hose could include an adapter or other structure to engage the connectors 1330, 1340.

Examples of materials from which mouthpieces 1334, 1344 could be made are disclosed elsewhere herein. The mouthpieces 1334, 1344 could be integrated with the hoses 1332, 1342 or attached to the hoses. Threaded engagements, friction fit engagements, magnetic engagements and/or other types of engagements could be used.

In FIG. 13, the mouthpiece 1334 is in fluid communication with the channel provided by the stem 1310, through a further channel that is provided by the hose 1332 and the manifold 1320. In an embodiment with multiple mouthpieces, the mouthpieces 1334, 1344 are in fluid communication with the channel provided by the stem 1310, through respective further channels that are provided by the hoses 1332, 1342 and the manifold 1320.

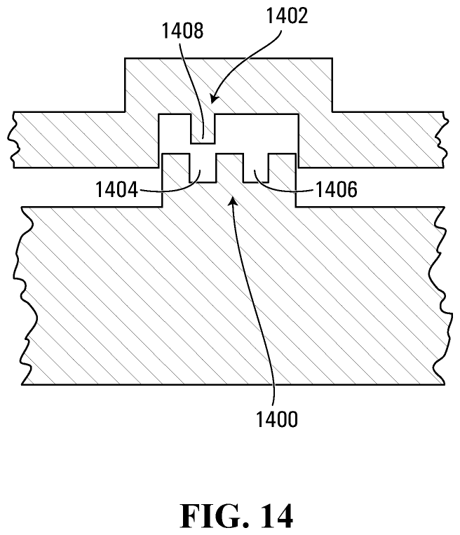

Several embodiments herein reference chamber engagement structures. FIG. 14 is a cross-sectional and partially exploded view of an example of engagement structures in a vaporization device. FIG. 14 illustrates an engagement structure 1400 and a complementary engagement structure 1402. Engagement structures could be used with replaceable or reconfigurable secondary chambers in a vaporization device. These engagement structures could be useful for restricting a vaporization device to a particular model or type of chamber or cartridge. Engagement structures could also or instead be useful as an assembly aid, to ensure that chambers or cartridges are assembled or installed properly. Further, the engagement structure for a chamber or cartridge could include or provide an indicator of the vaporization substance stored in the chamber or cartridge, and/or a type of the chamber or cartridge. A vaporization device could read this indicator to determine the type of vaporization substance, chamber, and/or cartridge. For example, some chambers or cartridges could include one or more auxiliary heaters and/or one or more active coolers, and a vaporization device could adapt power supply and/or control to a chamber or cartridge according to chamber or cartridge type.

In some embodiments, the engagement structure 1402 could be provided on the base of a chamber, at the point where the base contacts and/or engages with a battery compartment. Engagement structure 1400 could be provided on the battery compartment, at the point where the battery compartment contacts and/or engages with the base. In a specific example, referring to FIG. 4, the engagement structure 1402 could be provided on or toward the bottom of the base 406 in the view shown in FIG. 4, and the engagement structure 1400 could be provided on or toward the top of the battery compartment 408. When the base 406 and the battery compartment 408 are engaged, the engagement structures 1400, 1402 are also engaged. However, in general, other implementations are possible, such as providing engagement structures on stems, caps and/or other components of a vaporization device.

In the embodiment illustrated in FIG. 14, the engagement structure 1400 is sized to engage with the complementary engagement structure 1402. Therefore, only components with structures similar to the engagement structure 1400 will be able to couple to components containing the engagement structure 1400.

The engagement structure 1400 includes notches 1404 and 1406, and the complementary engagement structure 1402 includes a protrusion 1408. The protrusion 1408 could include a conductive pin and the notches 1404 and 1406 could include contacts, for example, to provide for detection of an installed chamber or cartridge and/or an installed chamber or cartridge type. Other embodiments are also contemplated, and the notches 1404 and 1406 could include pressure sensors or another type of sensor to detect the presence of a protrusion 1408.

Engagement structures that are similar to or different from the examples shown in FIG. 14 could be more specific to particular types of chambers. One or more engagement structures on an apparatus such as a vaporization device could mechanically restrict chambers, cartridges, and/or other components to only specific types. An engagement structure could include one or more features, such as one or more protrusions and/or one or more grooves, with size(s), shape(s), and/or positions to mate only with a particular type of cooperating component that has one or more complementary features. This type of physical or mechanical specificity could be used, for example, to restrict a vaporization device to use with only certain types of chambers or cartridges, which could provide a measure of control over the particular vaporization substances that are available for vaporization by a vaporization device. Certain chambers or cartridges could be restricted to certain vaporization devices, or to certain positions in a multi-chamber or multi-cartridge vaporization device, which could have regulators, power supply terminals, and/or other features that are specially adapted for those chambers or cartridges, for example.

As noted above, engagement structures need not have only a physical function such as controlling correct placement or alignment of a chamber and/or other component or limiting chambers and/or other components to particular types. Engagement structures on different chambers could have different sizes and/or patterns of conductive pins, for example, to enable a vaporization device to detect the type(s) of chambers that have been installed and adapt power and/or control accordingly.

In the example of FIG. 14, the presence of the protrusion 1408 aligned with the notch 1404 and the lack of a protrusion aligned with the notch 1406 could provide information regarding an installed chamber. This information could include the type of vaporization substance stored by a chamber, which could be used by a controller, in a base of a vaporization device, for example, to control the voltage, current, and/or power supplied to an atomizer, an auxiliary heater, and/or an active cooler. One or more regulators could also or instead be controlled based on the type of vaporization substance stored by a chamber or cartridge.

Each different type of chamber or cartridge that is compatible with a vaporization device could have a unique engagement structure. The two notches 1404 and 1406 in FIG. 14 can detect a maximum of four different types of chambers or cartridges, including those with no protrusions, those with two protrusions, those with only one protrusion 1408 as shown, and those with only one protrusion that corresponds to notch 1406. However, engagement structures with more or fewer notches could be used to different numbers of chamber types.

The protrusions and notches illustrated in FIG. 14 are provided by way of example only. Other arrangements, sizes, and shapes of engagement structures that might or might not include protrusions and/or grooves are also contemplated. Although described above primarily in the context of chambers, engagement structures could also or instead be used in conjunction with cartridges and/or other components. Engagement structures are also not in any way limited to localized structures at certain locations on or in an apparatus or component. Different types of chamber or cartridge could have different shapes that will only fit into compartments in a vaporization device that have a complementary shape, for example.

The foregoing description relates primarily to apparatus embodiments. Other embodiments, including methods, are also contemplated.

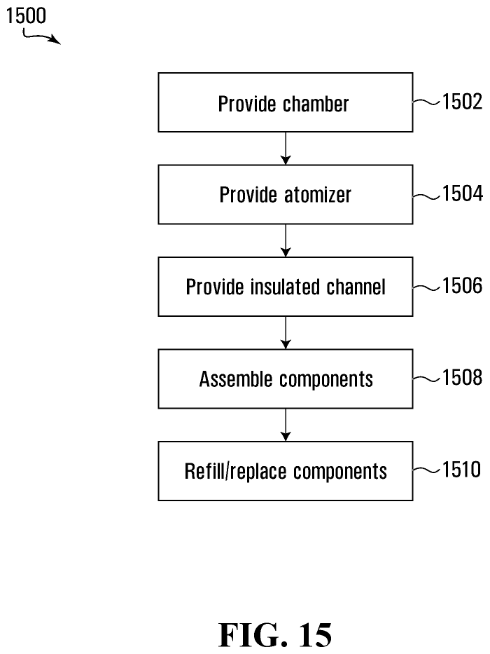

FIG. 15, for example, is a flow diagram illustrating a method 1500 according to an embodiment. The example method 1500 involves an operation 1502 of providing a chamber to store a vaporization substance, an operation 1504 of providing an atomizer to generate vapor from the vaporization substance by heating the vaporization substance, and an operation 1506 of providing an insulated channel to carry the vapor away from the atomizer.

These operations 1502, 1504, and 1506 are shown separately for illustrative purposes, but need not be separate operations in all embodiments. For example, a vaporization device or cartridge could include an atomizer, and could also be sold with a vaporization substance chamber and an insulated channel. A vaporization device, or components thereof, could potentially be provided separately from chambers, which could be purchased separately, for example. Some chambers could be provided with a vaporization device, while others could be sold separately.

A chamber, atomizer, and/or insulated channel could be provided at 1502, 1504, 1506 by actually manufacturing these components. Any of these components, and/or other components, could instead be provided by purchasing or otherwise acquiring the components from one or more suppliers.

At least some components or parts thereof could be provided in different ways. Different cartridge parts, such as chambers, bases, caps, atomizers, and/or stems, for example, could be provided by manufacturing one or more parts and purchasing one or more other parts, or by purchasing different parts from different suppliers.

In some embodiments, components such as the atomizer provided at 1504 and the insulated channel provided at 1506, and possibly the chamber provided at 1502, are provided in the form of a pre-assembled vaporization device. In other embodiments, components are not necessarily assembled. FIG. 15 therefore also illustrates an operation 1508 of assembling components. This could involve, for example, arranging the atomizer in fluid communication with a chamber and/or the insulated channel, such as by installing the atomizer, the insulated channel, and/or the chamber in a vaporization device or cartridge.

One or more components, such as a chamber, could be refilled or replaced as shown at 1510.

The example method 1500 is illustrative of one embodiment. Examples of various ways to perform the illustrated operations, additional operations that may be performed in some embodiments, or operations that could be omitted in some embodiments, could be inferred or apparent from the description and drawings, for example. Further variations may be or become apparent.

For example, providing an insulated channel at 1506 could involve providing, as the insulated channel, a channel that includes a thermally insulative material.

Another option for providing an insulated channel at 1506 involves providing a channel and providing a thermal insulator for the channel. Providing a thermal insulator for the channel could involve providing, and possibly installing or otherwise arranging, the thermal insulator at an inside part of the channel and/or at an outside part of the channel.

Providing a thermal insulator could also or instead involve providing, as the thermal insulator, a removable thermal insulator.

The thermal insulator could be provided as a coating on the channel.

A method based on the method 1500 could involve providing a cooler to cool the vapor. The cooler could be provided, and possibly installed or otherwise arranged, in fluid communication with the insulated channel, Providing a cooler could involve providing an active cooler such as a thermoelectric cooler, and/or providing a passive cooler such as a heat sink.

Providing a heat sink could involve providing a removable heat sink element, such as a removable heat sink element that is attachable, by a releasable coupling or magnetically for example, to an apparatus that includes the chamber, the atomizer, and the insulated channel.

In an embodiment, providing a cooler involves providing a heat exchanger to transfer heat from the vapor to a heat sink. The heat sink could be or include a material inside the heat exchanger.

In embodiments that involve a heat sink, the heat sink could be or include air or a liquid, for example.

A cooler could be provided inside the insulated channel.

A method based on the method 1500 could involve providing a mouthpiece in fluid communication with the insulated channel. Providing a mouthpiece could involve providing a mouthpiece that is releasably attachable, by a threaded engagement or by a friction fit engagement for example, to an apparatus that includes the chamber, the atomizer, and the insulated channel.

A mouthpiece could be indirectly in fluid communication with the insulated channel through a further channel.

In some embodiments, a method involves providing multiple mouthpieces in fluid communication with the insulated channel through respective further channels.

A method based on the method 1500 could involve providing a heater, in fluid communication with the atomizer, to heat the vapor, and/or providing a heater to heat the insulated channel.

In general, a method based on the method 1500 could involve providing a heater to heat one or more components of an apparatus that includes the chamber, the atomizer, and the insulated channel. The one or more components of the apparatus could include any one or more of: the insulated channel, and a regulator to control movement of the vaporization substance from the chamber to the atomizer.

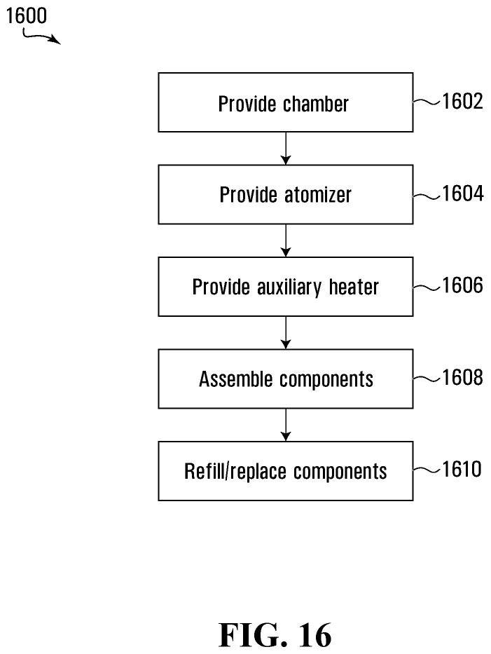

FIG. 16 is a flow diagram illustrating a method 1600 according to another embodiment. The example method 1600, like the example method 1500 in FIG. 15, involves an operation 1602 of providing a chamber to store a vaporization substance and an operation 1604 of providing an atomizer to generate vapor from the vaporization substance by heating the vaporization substance. The example method 1600 also includes an operation 1606 of providing an auxiliary heater, separate from a vaporization heater in the atomizer. Like the example method 1500 in FIG. 15, the example method 1600 also includes an operation 1608 of assembling components, and an operation 1610 of refilling and/or replacing one or more components. The operations 1602, 1604, 1608, 1610 could be similar to the operations 1502, 1504, 1508, 1510 discussed in detail above with reference to FIG. 15.

The operation 1608 could involve, for example, arranging an atomizer in fluid communication with a chamber and/or a channel, such as by installing the atomizer, the channel and/or the chamber in a vaporization device or cartridge. The operation 1608 could further involve arranging an auxiliary heater, in fluid communication with an atomizer in some embodiments.

The example method 1600, like the example method 1500, is an illustrative and non-limiting example. Various ways to perform the illustrated operations, additional operations that may be performed in some embodiments, or operations that could be omitted in some embodiments, could be inferred or apparent from the description and drawing or otherwise be or become apparent. Other variations of methods associated with manufacturing or otherwise producing an such as a chamber, a cartridge, or a vaporization device may be or become apparent.

For example, providing an auxiliary heater at 1606 could involve providing a vapor heater, and possibly installing or otherwise arranging the vapor heater in fluid communication with the atomizer, to heat the vapor. Providing an auxiliary heater at 1606 could also or instead involve providing a component heater to heat one or more components of an apparatus that includes the chamber, the atomizer, and the auxiliary heater. The one or more components of the apparatus could include any one or more of: a channel that is in fluid communication with the atomizer to carry the vapor away from the atomizer, and a regulator to control movement of the vaporization substance from the chamber to the atomizer.

A method based on the method 1600 could include providing a cooler, in fluid communication with the atomizer in some embodiments, to cool the vapor. Providing a cooler could involve providing an active cooler such as a thermoelectric cooler, and/or providing a passive cooler such as a heat sink.

Providing a heat sink could involve providing a removable heat sink element, which could involve providing a removable heat sink element that is attachable, by a releasable coupling or magnetically for example, to an apparatus that includes the chamber, the atomizer, and the auxiliary heater.

Providing a cooler could involve providing a heat exchanger to transfer heat from the vapor to a heat sink. The heat sink could be or include a material inside the heat exchanger.

In some embodiments, a heat sink could be or include air and/or a liquid.

Providing a cooler could involve providing, and possibly installing or otherwise arranging, the cooler inside a channel that is in fluid communication with the atomizer to carry the vapor away from the atomizer.

A method based on the method 1600 could involve providing a mouthpiece in fluid communication with the atomizer. Providing a mouthpiece could involve providing a mouthpiece that is releasably attachable, by a threaded engagement or by a friction fit engagement for example, to an apparatus that includes the chamber, the atomizer, and the auxiliary heater.

A method could involve providing a channel in fluid communication with the atomizer, in which case the mouthpiece could be indirectly in fluid communication with the atomizer through the channel and a further channel.

In an embodiment, a method includes providing multiple mouthpieces in fluid communication with the atomizer through respective channels.

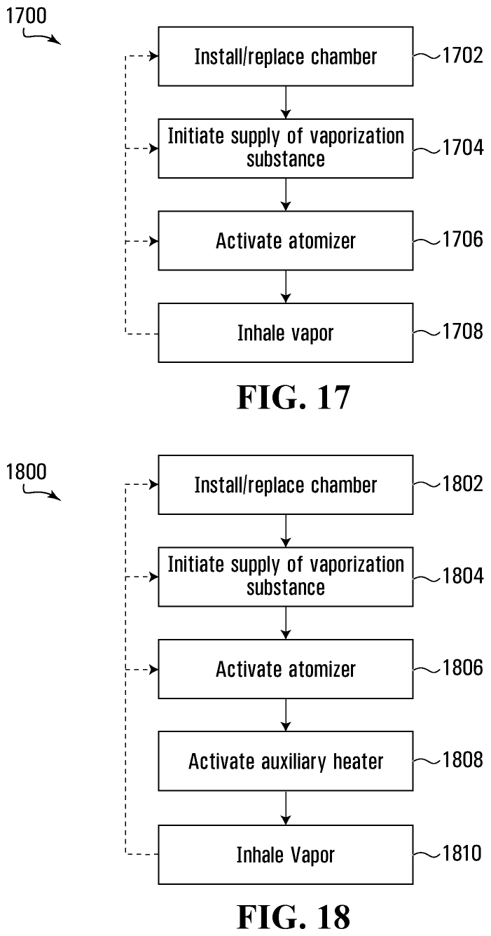

User methods are also contemplated. FIGS. 17 and 18 are flow diagrams illustrating methods 1700 and 1800 according to embodiments.

The example method 1700 involves an optional operation 1702 of installing or replacing a chamber. A user need not necessarily install or replace a chamber every time a vaporization substance is to be vaporized. The example method 1700 also involves an operation 1704 of initiating supply of a vaporization substance from the chamber to an atomizer, and an operation 1706 of activating the atomizer. These operations could involve operating one or more input devices such as a control button or switch or even just inhaling on a mouthpiece. The operations at 1704, 1706, 1708 are shown separately in FIG. 17 solely for illustrative purposes, and need not necessarily be separate operations.

Similarly, inhaling vapor through an insulated channel is represented separately at 1708, but in some embodiments inhaling on a mouthpiece initiates vaporization substance flow and vaporization.

The example method 1800 in FIG. 18 involves an optional operation 1802 of installing or replacing a chamber, an operation 1804 of initiating supply of a vaporization substance from the chamber to an atomizer, an operation 1806 of activating the atomizer, an operation 1808 of activating an auxiliary heater, and an operation 1810 of inhaling vapor. The operations 1802, 1804, 1806, 1810 could be similar to the operations 1702, 1704, 1706, 1708 of FIG. 17. The operation 1808 of activating an auxiliary heater could involve operating user input devices or inhaling on a mouthpiece at 1810.

The dashed arrows in FIG. 17 and FIG. 18 illustrate that multiple doses of a vaporization substance could be vaporized, and that a vaporization substance could be changed by installing or replacing a chamber at 1702, 1802.

The example methods 1700, 1800 are illustrative and non-limiting examples. Various ways to perform the illustrated operations, additional operations that may be performed in some embodiments, or operations that could be omitted in some embodiments, could be inferred or apparent from the description and drawing or otherwise be or become apparent.

It should be appreciated that the drawings and description herein are intended solely for illustrative purposes, and that the present invention is in no way limited to the particular example embodiments explicitly shown in the drawings and described herein.

What has been described is merely illustrative of the application of principles of embodiments of the present disclosure. Other arrangements and methods can be implemented by those skilled in the art. Thermal insulation and auxiliary heating, for example, are not necessarily mutually exclusive. Thermal insulation of one or more components could be implemented in a vaporization device that also includes one or more auxiliary heaters.

Illustrative embodiments have been described with reference to specific features and examples, various modifications and combinations can be made thereto without departing from the invention. The description and drawings are, accordingly, to be regarded simply as an illustration of some embodiments of the invention as defined by the appended claims, and are contemplated to cover any and all modifications, variations, combinations or equivalents that fall within the scope of the present invention. Therefore, although embodiments and potential advantages have been described by way of example in detail, various changes, substitutions and alterations can be made herein without departing from the invention as defined by the appended claims. Moreover, the scope of the present application is not intended to be limited to the particular embodiments of any process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

1. An apparatus comprising:
a chamber to store a vaporization substance;
an atomizer, in fluid communication with the chamber, to generate vapor from the vaporization substance by heating the vaporization substance;
an insulated channel, in fluid communication with the atomizer, to carry the vapor away from the atomizer,
wherein the insulated channel comprises a thermal insulator located at an inside part of the insulated channel.

2-5. (canceled)

6. The apparatus of claim 1, wherein the thermal insulator is removable.

7. The apparatus of claim 1, wherein the thermal insulator comprises a coating on the insulated channel.

8. (canceled)

9. The apparatus of claim 1, further comprising:
a cooler to cool the vapor, wherein the cooler comprises an active cooler.

10-14. (canceled)

15. The apparatus of claim 1, further comprising:
a cooler to cool the vapor,
wherein the cooler comprises a passive cooler,
wherein the passive cooler comprises a heat sink,
wherein the heat sink comprises a removable heat sink element,
wherein the removable heat sink element is coupled to the apparatus by a releasable coupling or magnetically.

16-17. (canceled)

18. The apparatus of claim 1, further comprising:
a cooler to cool the vapor,
wherein the cooler comprises a heat exchanger to transfer heat from the vapor to a heat sink,
wherein the heat sink comprises a material inside the heat exchanger,
wherein the heat sink comprises air or a liquid.

19-27. (canceled)

28. The apparatus of claim 1, comprising:
a heater to heat the insulated channel.

29. (canceled)

30. The apparatus of claim 1, comprising:
a heater to heat one or more components of the apparatus,
wherein the one or more components of the apparatus comprise any one or more of: the insulated channel, and a regulator to control movement of the vaporization substance from the chamber to the atomizer.

31. An apparatus comprising:
a chamber to store a vaporization substance;
an atomizer, in fluid communication with the chamber, to generate vapor from the vaporization substance by heating the vaporization substance;
an auxiliary heater,
wherein the auxiliary heater comprises:
a vapor heater to heat the vapor that is generated by the atomizer; or
a component heater to heat one or more components of the apparatus, wherein the one or more components of the apparatus comprise any one or more of: a channel that is in fluid communication with the atomizer to carry the vapor away from the atomizer, and a regulator to control movement of the vaporization substance from the chamber to the atomizer.

32-35. (canceled)

36. The apparatus of claim 31, further comprising:
a cooler to cool the vapor,
wherein the cooler comprises an active cooler.

37-41. (canceled)

42. The apparatus of claim 31, further comprising:
a cooler to cool the vapor,
wherein the cooler comprises a passive cooler,
wherein the passive cooler comprises a heat sink,
wherein the heat sink comprises a removable heat sink element,
wherein the removable heat sink element is coupled to the apparatus by a releasable coupling or magnetically.

43-53. (canceled)

54. A method comprising:
providing a chamber to store a vaporization substance;
providing an atomizer, in fluid communication with the chamber, to generate vapor from the vaporization substance by heating the vaporization substance;
providing an insulated channel in fluid communication with the atomizer, to carry the vapor away from the atomizer,
wherein providing an insulated channel comprises providing a thermal insulator at an inside part of the channel.

55-58. (canceled)

59. The method of claim 54, wherein providing a thermal insulator comprises providing as the thermal insulator a removable thermal insulator.

60. The method of claim 54, wherein providing a thermal insulator comprises providing as the thermal insulator a coating on the insulated channel.

61. (canceled)

62. The method of claim 54, further comprising:
providing a cooler to cool the vapor,
wherein providing a cooler comprises providing an active cooler.

63-67. (canceled)

68. The method of claim 54, further comprising:
providing a passive cooler to cool the vapor,
wherein providing a passive cooler comprises providing a heat sink,
wherein providing a heat sink comprises providing a removable heat sink element,
wherein providing a removable heat sink element comprises providing a removable heat sink element that is attachable, by a releasable coupling or magnetically, to an apparatus that comprises the chamber, the atomizer, and the insulated channel.

69-70. (canceled)

71. The method of claim 54, further comprising:
providing a cooler to cool the vapor,
wherein providing a cooler comprises providing a heat exchanger to transfer heat from the vapor to a heat sink,
wherein the heat sink comprises a material inside the heat exchanger,
wherein the heat sink comprises air or a liquid.

72-80. (canceled)

81. The method of claim 54, comprising:
providing a heater to heat the insulated channel.

82. (canceled)

83. The method of claim 54, comprising:
providing a heater to heat one or more components of an apparatus that comprises the chamber, the atomizer, and the insulated channel.
wherein the one or more components of the apparatus comprise any one or more of: the insulated channel, and a regulator to control movement of the vaporization substance from the chamber to the atomizer.

84. A method comprising:
providing a chamber to store a vaporization substance;
providing an atomizer, in fluid communication with the chamber, to generate vapor from the vaporization substance by heating the vaporization substance;
providing an auxiliary heater,
wherein providing an auxiliary heater comprises providing a vapor heater to heat the vapor, or providing an auxiliary heater comprises providing a component heater to heat one or more components of an apparatus that comprises the chamber, the atomizer, and the auxiliary heater, wherein the one or more components of the apparatus comprise any one or more of: a channel that is in fluid communication with the atomizer to carry the vapor away from the atomizer, and a regulator to control movement of the vaporization substance from the chamber to the atomizer.

85-88. (canceled)

89. The method of claim 84, further comprising:
providing a cooler to cool the vapor,
wherein providing a cooler comprises providing an active cooler.

90-94. (canceled)

95. The method of claim 84, further comprising:
providing a passive cooler to cool the vapor,
wherein providing a passive cooler comprises providing a heat sink,
wherein providing a heat sink comprises providing a removable heat sink element,
wherein providing a removable heat sink element comprises providing a removable heat sink element that is attachable, by a releasable coupling or magnetically, to an apparatus that comprises the chamber, the atomizer, and the auxiliary heater.

96-106. (canceled)

107. A method of use of the apparatus of claim 1, the method comprising:
initiating vaporization of the vaporization substance to produce a vapor;
inhaling the vapor through the insulated channel.

108. A method of use of the apparatus of claim 31, the method comprising:
initiating vaporization of the vaporization substance to produce a vapor;
initiating the auxiliary heater;
inhaling the vapor.
